(19) United States
(12) Patent Application Publication
(10) Pub. No.: US 2022/0340629 A1
(43) Pub. Date: Oct. 27, 2022

(54) Myosin Derived Peptides and Related Compounds with Anticoagulant Activities

(71) Applicants: THE SCRIPPS RESEARCH INSTITUTE, La Jolla, CA (US); SHANGPHARMA INNOVATION, South San Francisco, CA (US)

(72) Inventors: John H. Griffin, Del Mar, CA (US); Hiroshi Deguchi, San Diego, CA (US); Weijun Shen, San Diego, CA (US); Sam Lear, Del Mar, CA (US); Elsa Pflimlin, Koetzingue (FR); Yvonne Marie Angell, San Carlos, CA (US); Mike Lizarzaburu, Pacifica, CA (US)

(21) Appl. No.: 17/824,186

(22) Filed: May 25, 2022

Related U.S. Application Data
(63) Continuation of application No. 17/630,559, filed on Jan. 1, 0001.
(60) Provisional application No. 62/880,463, filed on Jul. 30, 2019.

(51) Int. Cl. C07K 14/47; A61K 38/17; A61K 47/54

(52) U.S. Cl. CPC C07K 14/4716; A61K 38/1719; A61K 47/543

(57) ABSTRACT

The present invention provides myosin derived peptides, variants and derivative compounds that are anti-coagulant. Also provided in the invention are antibodies that specifically target the peptides or epitopes presented thereon. Further provided in the invention are methods directed to using the anti-coagulant compounds described herein in therapeutic applications, e.g., inhibiting myosin-supported prothrombin activation to reduce the risk of acute trauma coagulopathy in post-trauma patients or inhibiting cardiac myosin-supported prothrombin activation to reduce the risk of coronary thrombosis.

### CROSS-REFERENCE TO RELATED APPLICATIONS

The subject patent application is a continuation of U.S. patent application Ser. No. 17/630,559 (filed Jan. 27, 2022; now pending), which is a § 371 U.S. national phase filing of PCT International Patent Application No. PCT/US2020/043878 (filed Jul. 28, 2020; now expired), which claims the benefit of priority to U.S. Provisional Patent Application No. 62/880,463 (filed Jul. 30, 2019; now expired). The full disclosures of the priority applications are incorporated herein by reference in their entirety and for all purposes.

### STATEMENT OF GOVERNMENT SUPPORT

This invention was made with government support under grant numbers HL021544 and HL133728 awarded by the National Institutes of Health. The government has certain rights in the invention.

### BACKGROUND OF THE INVENTION

Skeletal muscle myosin is a dimer of heterotrimers, each trimer comprising a regulatory light chain (RLC), an essential light chain (ELC), and a heavy chain (HC). It was discovered that skeletal muscle myosin has potent procoagulant and prothrombotic activity, providing a novel paradigm regarding how blood clotting may occur following events such as an acute trauma where major disruptions of coagulation (coagulopathy) often occurs. Infusion of skeletal muscle myosin reduced blood loss in an acquired hemophilia A mouse bleeding model, thereby evidencing myosin's in vivo prohemostatic activity. Mechanistic studies showed that myosin's potent prothrombotic activity involved enhancing thrombin generation due to myosin's ability to bind coagulation factors Xa and Va which accelerates prothrombin activation. However, detailed molecular mechanisms for myosin's binding to coagulation factors have not been established.

Myosin circulates in human plasma, and may contribute significantly more to the procoagulant potential of acute trauma patient plasmas than to that of healthy controls' plasma. By reducing thrombin generation, inhibition of the prothrombotic activity of myosin can lead to improved prognosis and ameliorated symptoms of disorders associated with myosin induced hypercoagulable sates. For example, inhibiting myosin-supported prothrombin activation can reduce the risk of acute trauma coagulopathy in post-trauma patients.

There is a need in the art for safe and effective means to reduce excess thrombin generation by inhibiting procoagulant activity of myosin. The present invention addresses this and other unmet needs in the art.

### SUMMARY OF THE INVENTION

In one aspect, the present invention provides myosin derived anti-coagulant peptides and related compounds. Typically, the peptides and related compounds possess an inhibitory activity on myosin mediated prothrombin activation, which can be measured via various assays exemplified herein. Some of the anti-coagulant peptides and related compounds of the invention are derived from human skeletal myosin, e.g., MYH2 as exemplified herein. In some embodiments, the anti-coagulant peptides and related compounds of the invention contain the sequence as shown in any one of SEQ ID NOs:10, 23 and 24, or a substantially identical or conservatively modified sequence thereof. In some of these embodiments, the anti-coagulant peptides and related compounds contain the sequence as shown in any one of SEQ ID NOs:16, 21, 22 and 28-96, or a substantially identical or conservatively modified sequence thereof.

In some embodiments, relative to the original or wildtype myosin chain sequences, the anti-coagulant peptides and related compounds of the invention contain one or more substitutions with non-natural amino acid residues, amino acid analogs or D-amino acid residues. In some embodiments, the anti-coagulant peptides and related compounds of the invention further contain an engineered disulfide bond or hydrocarbon bond. In some embodiments, the anti-coagulant peptides and related compounds of the invention further contain an N-terminal acylation. In some embodiments, the anti-coagulant peptides and related compounds of the invention further contain a C-terminal poly-lysine tail that consists of about 4 to about 8 D-lysine or L-lysine residues.

In some embodiments, the anti-coagulant peptides and related compounds of the invention further contain a chemical moiety (staple) that is attached to one or two residues in the sequence of the peptide. In some of these embodiments, the one or two residues are internal residues in the sequence of the peptide. In some other embodiments, the chemical moiety is attached to the N-terminal or the C-terminal residue of the peptide. In some embodiments, the chemical moiety is further conjugated to a lipid moiety.

In some related aspects, the invention provides pharmaceutical compositions. These compositions typically contain a therapeutically effective amount of a myosin derived anti-coagulant peptide or related compound described herein, optionally also a pharmaceutically acceptable carrier. Also provided in the invention are polynucleotide sequences that encode the anti-coagulant peptides and related compounds of the invention.

In another aspect, the invention provides derivative compounds of a myosin derived anti-coagulant peptide, as well as pharmaceutical compositions that contain such a derivative compound and a pharmaceutically acceptable carrier. These derivative compounds possess an inhibitory activity on myosin mediated prothrombin activation, which can be measured via one or more assays exemplified herein. Some of these compounds contain one or more modifications of a variant of SEQ ID NO:24 (HC816-835) or SEQ ID NO:21 (HC061). In some embodiments, the variant contains deletion of one or more terminal or internal residues of SEQ ID NO:24. In some embodiments, the variant contains one or more amino acid substitutions in SEQ ID NO:24 with non-natural amino acid residues, amino acid analogs or D-amino acid residues. In some embodiments, the variant contains SEQ ID NO:24 with C-terminal additions of (1) an Nle residue and (2) a poly-lysine tail that consists of about 4 to about 8 L-lysine or D-lysine residues.

In some derivative compounds of the invention, the variant contains SEQ ID NO:24 that is modified by conjugating a chemical moiety (staple) to one or two residues in the sequence of the peptide. In some of these embodiments, the variant is modified by one or more of the following substitutions in SEQ ID NO:24: Cys⁴→Lys, Thr or Ser, Ser¹¹→Lys, Met¹³→Nle, and Asn¹⁴→N-MeN. In some embodiments, the chemical moiety is attached to an engineered Lys residue that substitutes for Ser¹¹ in SEQ ID NO:24. In some embodiments, the chemical moiety is KA21, KA10, KA26, KA18, KA19, KA3, KA24, KA23, KA27 or KA22 as shown in FIG. 16. In some of the derivative compounds, a lipid or fatty acid moiety is conjugated to the chemical moiety.

In still another aspect, the invention provides methods for treating or preventing undesired thrombosis in a subject. These methods entail administering to the subject a pharmaceutical composition that comprises a therapeutically effect amount of (1) a myosin derived anti-coagulant peptide that contains the sequence shown in any one of SEQ ID NOs:10, 23 and 24, or a substantially identical or conservatively modified sequence thereof or (2) a derivative compound of a myosin derived anti-coagulant peptide that contains one or more modifications of a variant of SEQ ID NO:24 (HC816-835) or SEQ ID NO:21 (HC061). In various embodiments, the undesired thrombosis to be treated or prevented is supported or mediated by myosin such as skeletal myosin or cardiac myosin. In some embodiments, the subject amenable for the therapeutic methods of the invention is an acute trauma patient. In some of these embodiments, the subject is one who has or is at risk of developing acute trauma coagulopathy. In some embodiments, the subject is one who has or is at risk of developing coronary thrombosis.

A further understanding of the nature and advantages of the present invention may be realized by reference to the remaining portions of the specification and claims.

### BRIEF DESCRIPTION OF THE DRAWINGS

FIG. 1. Screening for the inhibitory effects of myosin neck region peptides on myosin-enhanced or phospholipid-enhanced prothrombin activation by factors Xa and Va. Skeletal muscle myosin (2 nM, final) (A) or phospholipid vesicles (PC/PS, 80%/20% w/w) (4 μM, final) (B) were incubated with 19 myosin-derived peptides at 3 different peptide concentrations, 5μM , 25 μM and 100 μM, respectively, as indicated, with factor Va (5 nM, final), and factor Xa (0.2 nM, final) in TBS containing 0.5% BSA (TB SA) plus 5 mM CaCl₂ at room temperature. Thrombin generation was determined as described in Experimental procedures. 100% was the value for controls in the absence of added peptides. Each value represents the mean [±standard error of the mean (SEM)] of quadruplicate determinations.

FIG. 2. Inhibition of myosin-enhanced prothrombin activation by myosin peptides and binding of peptide HC796-835 to factor Xa. Varying concentrations of peptides HC781-810, HC 815-854, HC796-835, and RLC133-162 (0-100 μM) were incubated with factor Va (5 nM, final) and factor Xa (0.2 nM, final) in TB SA plus 5 mM CaCl₂ at room temperature in the presence of skeletal muscle myosin (2 nM, final) (A) or phospholipid vesicles (PC/PS, 80%/20% w/w) (4 μM, final) (B). Thrombin generation was initiated by the addition of prothrombin (0.75 μM) in TBSA containing 5 mM CaCl₂. The reaction was quenched by adding EDTA (10 mM final) at 10 min. Thrombin generation was determined as described in Experimental procedures. 100% was the value for controls in the absence of added peptides. (C) Peptide HC796-835 was immobilized in microtiter plate wells and the binding of Factor Xa to HC796-835 was determined as described in Experimental procedures. Each value represents the mean [±SEM] of triplicate determinations.

FIG. 3. Peptide screening for their inhibition of Tissue Factor-induced thrombin generation in human plasma. 19 myosin-derived peptides (100 μM, final) (A) or varying concentrations of peptide HC 796-835 (0-25 μM, final) (B) were assayed for their ability to inhibit thrombin generation in human plasma, as described in Experimental procedures. 100 was the value for controls in the absence of added peptides. Each value represents the mean [±SEM] of quadruplicate determinations.

FIG. 4. Inhibition of prothrombin activation by TFP or myosin peptides in the presence or absence of myosin. Varying concentrations of TFP (0-100 μM) (A) or peptides ELC129-144 (B), ELC138-157 (C) and ELC146-161 (D) (0-300 μM) were incubated with factor Va (5 nM, final) and factor Xa (0.2 nM, final) in TB SA plus 5 mM CaCl₂ at room temperature in the presence or absence of skeletal muscle myosin (2 nM, final). Thrombin generation was initiated by the addition of prothrombin (0.75 μM) in TB SA containing 5 mM CaCl₂. The reaction was quenched by adding EDTA (10 mM final) at 10 min. Thrombin formation was quantified by the rate of substrate (Pefa TH) hydrolysis. 100% was the value for controls in the absence of added peptides. Each value represents the mean [±SEM] of at least triplicate determinations.

FIG. 5 shows effect of several myosin HC derived peptides on prothrombin activation by factor Xa/factor Va/myosin in a purified system. Sequences of the peptides including replacement residues are indicated in the table below. N. norleucine (Nle); *: Lys(Alloc), #: Lys(ivDde), protecting groups removed and then stapled.

FIG. 6 shows IC50 for anticoagulant peptides in secondary in vitro assays in plasma. In vitro activity of the early lead peptides in plasma assays. Sequences of the peptides including replacement residues are indicated in the table below N. norleucine (Nle); *: Lys(Alloc), #: Lys(ivDde), protecting groups removed and then stapled.

FIG. 7 shows results of Alanine scan of peptide HC060 results. Amino acids replaced by alanine are indicated in red.

FIG. 8 shows six sites of protease cleavage in HC060 identified from metabolite identification when incubated in mouse plasma.

FIG. 9 shows effect of substitutions of important residues identified by alanine scanning with unnatural amino acids. Red: amino acid replaced by unnatural amino acid; abbreviations: 2Nal: 2-Napthylalanine; 3Pal: 3-pyridylalanine; Tha: 2-Thienylalanine; Cba: Cyclobutylalanine; A6c: homocycloleucine; Cha: cyclohexylalanine; Aib: Aminoisobutylalanine; N-MeA: N-Methyl alanine.

FIG. 10 shows activities of peptide derived from HC061 sequence stapled with K1 staple at various positions. Optimization of staple length was carried out using ‘simple’ staples K1, K1F, K1H and Al, before incorporation of the lipid moiety (e.g. K5). Scanning of stapling sites was carried out within the HC061 sequence was carried out using the K1 staple, to find the position most beneficial to activity.

FIG. 11 shows effect of lipid structure and position on conjugate activity.

FIG. 12 shows circular dichroism spectra study of peptides HC160 and HC095. The results indicate that K1 stapling in peptide HC160 leads to enhancement of helicity.

FIG. 13 shows examples of simple staple types/lengths (‘K1’ series and A1) and lipidated staple K5, which allows simultaneous fatty acid conjugation and stapling.

FIG. 14 shows examples of lipid structures illustrating variation in PEG linker length (blue) and fatty acid chain length (red) in addition to differing carboxylate placement.

FIG. 15 shows structures of various amino acid analogs that were used to substitute residues in the myosin derived anti-coagulant variant peptides.

FIG. 16 shows structures of various chemical moieties (staples) that were used to modify the myosin derived anti-coagulant variant peptides.

FIG. 17 shows inhibition of cardiac myosin-enhanced prothrombin activation by peptide HC796-835 (SEQ ID NO:21).

FIG. 18 shows inhibition of myosin-dependent prothrombin activation by murine antisera from mice immunized with SEQ ID NO:97 that was modified on either N-terminus or C-terminus by adding Cys-coupled-keyhole limpet hemocyanin for immunization purposes.

### DETAILED DESCRIPTION OF THE INVENTION

### I. Overview

Skeletal muscle myosin has potent prothrombotic and procoagulant activities. Mechanistic studies showed that myosin's procoagulant activity is based on its ability to enhance thrombin generation due to binding coagulation factors Xa and Va and accelerating prothrombin activation. The present invention is predicated in part on the discoveries by the present inventors that some myosin derived peptides have anti-coagulant functions. Specifically, as part of an effort to identify the binding sites on myosin for coagulation factors, myosin peptides were synthesized and assayed for their ability to inhibit myosin-enhanced prothrombin activation by factors Va and Xa. As detailed herein, the inventors initially screened 3 ELC peptides (SEQ ID NOs:1-3). Based on positive ELC peptide data, the inventors then screened 19 peptides (SEQ ID NOs:4-22) representing additional myosin sequences located in the myosin neck region where myosin's three polypeptides, the HC, RLC, and ELC are clustered. Data from these screening assays identified a myosin HC peptide sequence that binds factor Xa and inhibits myosin-enhanced prothrombinase activity. These findings support the hypothesis that myosin's neck region binds blood coagulation factor Xa and is responsible for myosin's ability to enhance thrombin generation.

As detailed herein, the 19 screened peptides contained 25-40 residues and overlapped by ˜5-10 amino acids. These peptides were tested for their inhibition of myosin-supported prothrombin activation by purified factor Xa, factor Va, and Ca⁺⁺ ions. Based on results from the studies, it was identified that ELC residues 129-138 (SEQ ID NO:23), are key for myosin's procoagulant activity. In addition, RLC residues 133-162 (SEQ ID NO:10) were also found to be important for myosin's specific procoagulant activity. The inventors further examined potential sites on the HC chain that are responsible for interaction with factors Xa and Va. It was found that Peptide HC796-835 (SEQ ID NO:21) derived from the heavy chain strongly inhibited myosin-enhanced prothrombin activation by factors Xa and Va but not phospholipid vesicle-enhanced prothrombin activation. This implies that this sequence in the myosin heavy chain interacts with factor Xa and/or factor Va. Further studies showed that residues 816-835 (SEQ ID NO:24) of skeletal muscle myosin heavy chain are responsible for binding factor Xa and that this binding is essential to myosin's procoagulant activity.

Additionally, the inventors undertook studies to identify and generate variant peptides and derivative compounds that are based on the anti-coagulant myosin peptide sequences described herein (e.g., SEQ ID NOs:21 and 24). As detailed herein, the variants and derivatives were obtained by rational design, a series of modifications and optimization, followed by various assays to identify compounds with desired structures and functional properties. A number of variant peptides or derivative compounds with improved biological activities and/or pharmaceutical properties were obtained.

In accordance with these discoveries, the invention provides novel anticoagulant agents that can selectively inhibit myosin's prothrombotic activities and to reduce thrombin generation. Also provided in the invention are antibodies that specifically target myosin epitopes that are responsible for its procoagulant activities. The invention additional provides therapeutic applications of the anti-coagulant compounds described herein for treating or preventing excessive coagulation or thrombosis in acute trauma patients.

Unless otherwise stated, the present invention can be performed using standard procedures, as described, for example in Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906); U.S. Pat. Nos. 4,965,343, and 5,849,954; Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998). The following sections provide additional guidance for practicing the compositions and methods of the present invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Oxford Dictionary of Biochemistry and Molecular Biology, Smith et al. (eds.), Oxford University Press (revised ed., 2000); Dictionary of Microbiology and Molecular Biology, Singleton et al. (Eds.), John Wiley & Sons (3PrdP ed., 2002); and A Dictionary of Biology (Oxford Paperback Reference), Martin and Hine (Eds.), Oxford University Press (4PthP ed., 2000). In addition, the following definitions are provided to assist the reader in the practice of the invention.

The singular terms “a,” “an,” and “the” include plural referents unless the context clearly indicates otherwise. Similarly, the word “or” is intended to include “and” unless the context clearly indicates otherwise.

As used herein, the term “amino acid” of a peptide refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The myosin derived anti-coagulant peptides of the invention encompass derivative or analogs which have be modified with non-naturally coding amino acids.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The term “conservatively modified variant” or “conservatively substituted variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, a “derivative” of a reference molecule (e.g., an anti-coagulant peptide disclosed herein) is a molecule that is chemically modified relative to the reference molecule while substantially retaining the biological activity. The modification can be, e.g., oligomerization or polymerization, modifications of amino acid residues or peptide backbone, cross-linking, cyclization, conjugation, fusion to additional heterologous amino acid sequences, or other modifications that substantially alter the stability, solubility, or other properties of the peptide.

The terms “decrease” , “reduced”, “reduction” , “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The term “engineered cell” or “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “epitope” as used herein refers to portions of a polypeptide (e.g., a myosin derived anti-coagulant peptide described herein) having antigenic or immunogenic activity in an animal, for example a mammal, for example, a human. An “immunogenic epitope,” as used herein, is defined as a portion of a protein that elicits an immune response in an animal, as determined by any method known in the art. The term “antigenic epitope,” as used herein, is defined as a portion of a protein to which an antibody can immunospecifically bind its antigen as determined by any method well known in the art. Immunospecific binding excludes non-specific binding but does not necessarily exclude cross-reactivity with other antigens. Antigenic epitopes need not necessarily be immunogenic. In the present invention, antigenic epitopes preferably contain a sequence of at least 4, at least 5, at least 6, at least 7, at least 8, at least 9, at least 10, at least 15, at least 20, at least 25, or between about 15 to about 30 amino acids contained within the amino acid sequence of a polypeptide of the invention. Certain polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Antigenic as well as immunogenic epitopes may be linear, i.e., be comprised of contiguous amino acids in a polypeptide, or may be three dimensional, i.e., where an epitope is comprised of non-contiguous amino acids which come together due to the secondary or tertiary structure of the polypeptide, thereby forming an epitope. As to the selection of peptides or polypeptides bearing an antigenic epitope (e.g., that contain a region of a protein molecule to which an antibody or T cell receptor can bind), it is well known in that art that relatively short synthetic peptides that mimic part of a protein sequence are routinely capable of eliciting an antiserum that reacts with the partially mimicked protein. See, e.g., Sutcliffe, J. G., et al., Science 219:660-666 (1983).

The term “fragment” refers to any peptide or polypeptide having an amino acid residue sequence shorter than that of a full-length polypeptide whose amino acid residue sequence is described herein. An isolated peptide of a myosin chain (e.g., human myosin HC, ELC or RLC) is shortened or truncated compared to its parent full-length polypeptide chain. Relative to a full length myosin chain, the anti-coagulant myosin peptides described herein typically contain one of the specific sequences exemplified herein, e.g., SEQ ID NOs:1-24. They can additionally contain deletions at the N-terminus and/or the C-terminus, as well as various substitutions or modifications as described herein.

The term “isolated” means the protein is removed from its natural surroundings. However, some of the components found with it may continue to be with an “isolated” protein. Thus, an “isolated polypeptide” is not as it appears in nature but may be substantially less than 100% pure protein.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Methods of alignment of sequences for comparison are well known in the art. Optimal alignment of sequences for comparison can be conducted, e.g., by the local homology algorithm of Smith and Waterman, Adv. Appl. Math. 2:482c, 1970; by the homology alignment algorithm of Needleman and Wunsch, J. Mol. Biol. 48:443, 1970; by the search for similarity method of Pearson and Lipman, Proc. Nat'l. Acad. Sci. USA 85:2444, 1988; by computerized implementations of these algorithms (GAP, BESTFIT, FASTA, and TFASTA in the Wisconsin Genetics Software Package, Genetics Computer Group, Madison, WI); or by manual alignment and visual inspection (see, e.g., Brent et al., Current Protocols in Molecular Biology, John Wiley & Sons, Inc. (ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

Unless otherwise specified, the terms “polypeptide” and “peptide” are used interchangeably herein (e.g., “myosin derived anti-coagulant peptides or polypeptide”) and to refer to a polymer of amino acid residues. They encompass both short oligopeptides (e.g., peptides with less than about 25 residues) and longer polypeptide molecules (e.g., polymers of more than about 25 or 30 amino acid residues). Typically, the anti-coagulant peptides (oligopeptides) or polypeptides of the invention can comprise from about 4 amino acid residues to about 100 or more amino acid residues in length. In some embodiments, the peptides or polypeptides comprise from about 8 amino acid residues to about 60 amino acid residues in length. The anti-coagulant peptides or polypeptides of the invention include naturally occurring amino acid polymers and non-naturally occurring amino acid polymer, as well as amino acid polymers in which one or more amino acid residue is an artificial chemical mimetic of a corresponding naturally occurring amino acid. Unless otherwise indicated, a particular polypeptide sequence also implicitly encompasses conservatively modified variants thereof.

As used herein, the term “peptide mimetic” or “peptidomimetic” refers to a derivative compound of a reference peptide (e.g., an anti-coagulant polypeptide disclosed herein) that biologically mimics the peptide's functions. Typically, the peptidomimetic derivative of a myosin derived anti-coagulant peptide of the invention has at least 50%, at least 75% or at least 90% of the biological activities (e.g., inhibition of prothrombin activation) of the reference peptide (e.g., SEQ ID NOs:1-24, 97 and 98).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term “orthologs” or “homologs” refers to polypeptides that share substantial sequence identity and have the same or similar function from different species or organisms. For example, each of the 3 myosin chains from human, rabbit, rat, mouse and many other animal species are orthologs due to the similarities in their sequences and functions.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The term “agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

Myosin is a highly conserved, ubiquitous family of protein found in all eukaryotic cells, where it provides the motor function for diverse movements such as cytokinesis, phagocytosis, and muscle contraction. All myosins contain an amino-terminal motor/head domain and a carboxy-terminal tail domain. Due to the extensive number of different molecules identified to date, myosins have been divided into seven distinct classes based on the properties of the head domain. One such class, class II myosins, consists of the conventional two-headed myosins that form filaments and are composed of two myosin heavy chain (HC) subunits and four myosin light chain subunits (2 essential light chain (ELC) subunits and 2 regulatory light chain (RLC) subunits). The myosin heavy chain subunit contains the ATPase activity providing energy that is the driving force for contractile processes mentioned above, and numerous myosin HC isoforms exist in vertebrates to carry out this function. Exemplary sequences of the heavy chain (HC), essential light chain (ELC) and regulatory light chain (RLC) of human skeletal myosin are shown in SEQ ID NOs:25-27, respectively.

Myosin-II isoforms are the major contractile proteins in muscle and also play several crucial roles in non-muscle contractility. Myosin-II molecules contain two motor domains and assemble into bipolar filaments. A number of genes encoding the myosin heavy chain isoforms (MYH cluster genes) have been identified. These include MYH1 (skeletal muscle, adult), MYH2 (skeletal muscle, adult), MYH3 (skeletal muscle, embryonic), MYH4 (skeletal muscle), MYH6 (cardiac muscle), MYH7 (cardiac muscle), MYH7B (cardiac muscle), MYH8 (skeletal muscle, perinatal), MYH9 (non-muscle), MYH10 (non-muscle), MYH11 (smooth muscle), MYH13 (skeletal muscle), MYH14 (non-muscle), MYH15 and MYH16. In humans and mice, skeletal myosin heavy chain (MYH) genes are clustered on a single chromosome (17p in humans and 11 in mice).

The term subject or patient can include human or non-human animals. Thus, the methods and described herein are applicable to both human and veterinary disease and animal models. Preferred subjects are “patients,” i.e., living humans that are receiving medical care for a disease or condition. This includes persons with no defined illness who are being investigated for signs of pathology.

The phrase “substantially identical,” in the context of two nucleic acids or polypeptides, refers to two or more sequences or subsequences that have at least 75%, preferably at least 85%, more preferably at least 90%, 95% or higher nucleotide or amino acid residue identity, when compared and aligned for maximum correspondence, as measured using a sequence comparison algorithm such as those described below for example, or by visual inspection. Preferably, the substantial identity exists over a region of the sequences that is at least about 40-50 residues in length, preferably over a longer region than 50 amino acids, more preferably at least about 90-100 residues, and most preferably the sequences are substantially identical over the full length of the sequences being compared, such as the coding region of a nucleotide for example.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection. Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. Another indication that two nucleic acid sequences are substantially identical is that the two molecules hybridize to each other under stringent conditions. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA.

As used herein, the term “variant” refers to a molecule (e.g., a polypeptide or polynucleotide) that contains a sequence that is substantially identical to the sequence of a reference molecule. For example, the reference molecule can be a myosin derived anti-coagulant peptide (e.g., SEQ ID NOs:1-24) or a polynucleotide encoding the polypeptide. In some embodiments, the variant can share at least 50%, at least 70%, at least 80%, at least 90, at least 95%, at least 96%, at least 97%, at least 98%, at least 99% or more sequence identity with the reference molecule. In some other embodiments, the variant differs from the reference molecule by having one or more conservative amino acid substitutions. In some other embodiments, a variant of a reference molecule has altered amino acid sequences (e.g., with one or more conservative amino acid substitutions) but substantially retains the biological activity of the reference molecule (e.g., anti-coagulation). Conservative amino acid substitutions are well known to one skilled in the art.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

### III. Myosin Derived Anti-Coagulant Peptides

The invention provides skeletal muscle myosin chain derived anti-coagulant peptides and derivative compounds. The myosin chain derived anti-coagulant peptides include peptides derived from all three chains of skeletal myosin, including the heavy chain (HC), the essential light chain (ELC), and the regulatory light chain (RLC). Provided they possess activities in inhibiting myosin-supported prothrombin activation, anti-coagulant peptides and derivative compounds of the invention can encompass any portions, fragments or combination thereof of each of the 3 skeletal muscle myosin chains. Anti-coagulant activities of the peptides and derivative compounds described herein can be readily assessed with methods that are well known in the art or the assays exemplified herein. These include, e.g., assay for prothrombin activation and assay for thrombin generation in plasma. See, e.g., Deguchi et al., Blood 128, 1870-1878, 2016; Deguchi et al., Br. J. Haematol. 165, 409-412, 2014; Hemker et al., Thromb. Res. 131:3-11, 2013; and Tripodi, Clin. Chem. 62:5, 2016.

In some embodiments, the anti-coagulant peptides of the invention are derived from human skeletal muscle myosin. Sequences of human skeletal muscle myosin chains are well known and extensively characterized in the art. These include various isoforms of all three human skeletal muscle myosin chains. See, e.g., Saez et al., Nucleic Acids Res. 14:2951-2969, 1986; Karsch-Mizrachi et al., Nucleic Acids Res. 17:6167-6179, 1989; Hailstones et al., Mol. Cell. Biol. 10:1095-1104, 1990; Macera et al., Genomics 13:829-831, 1992; Aikawa et al., Circ. Res. 73:1000-1012, 1993; Rourke et al., J. Appl. Physiol. 97:1985-1991, 2004; Resnicow et al., Proc. Natl. Acad. Sci. USA 107:1053-1058, 2010; and Walklate et al., J. Exp. Biol. 219:168-174, 2016. Unless otherwise noted, the myosin chain sequences described herein are based on human skeletal muscle heavy chain isoform MYH2 (SEQ ID NO:25), essential light chain MYL1 (SEQ ID NO:26), and regulatory light chain MYLPF (SEQ ID NO:27). These sequences were characterized in the art. See, e.g., Weiss et al., Proc. Natl. Acad. Sci. U.S.A. 96:2958-2963, 1999; Weiss et al., J. Mol. Biol. 290:61-75, 1999; Seidel et al., Nucleic Acids Res. 15:4989, 1987; Seidel et al., Gene 66:135-146, 1988; Garfinkel et al., J. Biol. Chem. 257:11078-11086, 1982; and Sachdev et al., DNA Seq. 14:339-350, 2003.

In various embodiments, anti-coagulant peptides of the invention encompass residues of fragments of myosin chains that correspond to the neck region of skeletal muscle myosin. The neck region represents the portion of myosin's 3-dimentional structure where myosin's 3 chains are clustered and where factor Xa is bound to promote prothrombin activation by factors Xa/Va. As exemplified herein with peptide sequences shown in SEQ ID NOs:10, 21, 23 and 24, these peptides are able to specifically inhibit prothrombin activation and coagulant activities that are mediated or supported by myosin.

In some embodiments, skeletal muscle myosin chain derived anti-coagulant peptides of the invention comprises a sequence as set forth in SEQ ID NO:10, 23 or 24, a substantially identical sequence or a conservatively modified variant thereof. In some embodiments, the peptide has a sequence that is derived from human myosin RLC fragment sequence shown in SEQ ID NO:10. In some of these embodiments, the peptide comprises a sequence that includes the sequence shown in SEQ ID NO:10 or a conservatively substituted variant thereof. For example, the peptide can contain at least 30, 31, 32, 33, 34, 35, 40, 45 or more contiguous amino acid residues of SEQ ID NO:RLC encompassing SEQ ID NO:10. In some other embodiments, the peptide comprises a sequence that includes a N-terminally and/or a C-terminal truncation of SEQ ID NO:10 or a conservatively substituted variant thereof. For example, the peptide can contain a variant of SEQ ID NO:10 containing a deletion of 1, 2, 3, 4, 5, 6, 7, 8 or more residues at the N-terminus of SEQ ID NO:10, and/or a deletion of 1, 2, 3, 4, 5, 6, 7, 8 or more residues at the C-terminus of SEQ ID NO:10. Some exemplary peptides in these embodiments include, e.g.,

(SEQ ID NO: 46)
SQEEIKNMWAAFPPDVGGNVDYKNICYVITH,
(SEQ ID NO: 47)
SQEEIKNMWAAFPPDVGGNVDYKNICYVITHG,
(SEQ ID NO: 48)
SQEEIKNMWAAFPPDVGGNVDYKNICYVITHGD,
(SEQ ID NO: 49)
SQEEIKNMWAAFPPDVGGNVDYKNICYVITHGDA,
(SEQ ID NO: 50)
SQEEIKNMWAAFPPDVGGNVDYKNICYVITHGDAK,
(SEQ ID NO: 51)
FSQEEIKNMWAAFPPDVGGNVDYKNICYVITHG,
(SEQ ID NO: 52)
FSQEEIKNMWAAFPPDVGGNVDYKNICYVITHGD,
(SEQ ID NO: 53)
FSQEEIKNMWAAFPPDVGGNVDYKNICYVITHGDA,
(SEQ ID NO: 54)
FSQEEIKNMWAAFPPDVGGNVDYKNICYVITHGDAK,
(SEQ ID NO: 55)
RFSQEEIKNMWAAFPPDVGGNVDYKNICYVITHG,
(SEQ ID NO: 56)
RFSQEEIKNMWAAFPPDVGGNVDYKNICYVITHGD,
(SEQ ID NO: 57)
RFSQEEIKNMWAAFPPDVGGNVDYKNICYVITHGDA,
(SEQ ID NO: 58)
RFSQEEIKNMWAAFPPDVGGNVDYKNICYVITHGDAK,
(SEQ ID NO: 59)
DRFSQEEIKNMWAAFPPDVGGNVDYKNICYVITHG,
(SEQ ID NO: 60)
DRFSQEEIKNMWAAFPPDVGGNVDYKNICYVITHGD,
(SEQ ID NO: 61)
DRFSQEEIKNMWAAFPPDVGGNVDYKNICYVITHGDA,
(SEQ ID NO: 62)
DRFSQEEIKNMWAAFPPDVGGNVDYKNICYVITHGDAK,
(SEQ ID NO: 63)
FSQEEIKNMWAAFPPDVGGNVDYKNICYVITH,
(SEQ ID NO: 64)
RFSQEEIKNMWAAFPPDVGGNVDYKNICYVITH,
(SEQ ID NO: 65)
DRFSQEEIKNMWAAFPPDVGGNVDYKNICYVITH,
(SEQ ID NO: 66)
QEEIKNMWAAFPPDVGGNVDYKNICYVITHG,
(SEQ ID NO: 67)
QEEIKNMWAAFPPDVGGNVDYKNICYVITHGD,
(SEQ ID NO: 68)
QEEIKNMWAAFPPDVGGNVDYKNICYVITHGDA,
(SEQ ID NO: 69)
QEEIKNMWAAFPPDVGGNVDYKNICYVITHGDAK,

or a conservatively substituted variant thereof.

In some embodiments, skeletal muscle myosin chain derived anti-coagulant peptides of the invention contain a sequence that is derived from human myosin ELC fragment sequence shown in SEQ ID NO:23 (YEDFVEGLRV). In some of these embodiments, the peptide comprises a sequence that includes the sequence shown in SEQ ID NO:23 or a conservatively substituted variant thereof. For example, the peptide can contain at least 11, 12, 13, 14, 15, 16, 17, 18, 19, 20, 25, or more contiguous amino acid residues of SEQ ID NO:ELC encompassing SEQ ID NO:23. In some other embodiments, the peptide comprises a sequence that includes a N-terminally and/or a C-terminal truncation of SEQ ID NO:23 or a conservatively substituted variant thereof. For example, the peptide can contain a variant of SEQ ID NO:23 containing a deletion of 1, 2, 3, or more residues at the N-terminus of SEQ ID NO:23, and/or a deletion of 1, 2, 3, or more residues at the C-terminus of SEQ ID NO:23. Some exemplary peptides in these embodiments include, e.g.,

(SEQ ID NO: 70)
EFEQFLPMMQAISNNKDQATYEDFVEGLRV,
(SEQ ID NO: 71)
EQFLPMMQAISNNKDQATYEDFVEGLRV,
(SEQ ID NO: 72)
EQFLPMMQAISNNKDQATYEDFVEGLRV,
(SEQ ID NO: 73)
QFLPMMQAISNNKDQATYEDFVEGLRV,
(SEQ ID NO: 74)
FLPMMQAISNNKDQATYEDFVEGLRV,
(SEQ ID NO: 75)
LPMMQAISNNKDQATYEDFVEGLRV,
(SEQ ID NO: 76)
LPMMQAISNNKDQATYEDFVEGLRV,
(SEQ ID NO: 77)
MMQAISNNKDQATYEDFVEGLRV,
(SEQ ID NO: 78)
MQAISNNKDQATYEDFVEGLRV,
(SEQ ID NO: 79)
QAISNNKDQATYEDFVEGLRV,
(SEQ ID NO: 80)
AISNNKDQATYEDFVEGLRV,
(SEQ ID NO: 81)
ISNNKDQATYEDFVEGLRV,
(SEQ ID NO: 82)
SNNKDQATYEDFVEGLRV,
(SEQ ID NO: 83)
NNKDQATYEDFVEGLRV,
(SEQ ID NO: 84)
NKDQATYEDFVEGLRV,
(SEQ ID NO: 85)
KDQATYEDFVEGLRV,
(SEQ ID NO: 86)
DQATYEDFVEGLRV,
(SEQ ID NO: 87)
QATYEDFVEGLRV,
(SEQ ID NO: 88)
ATYEDFVEGLRV,
(SEQ ID NO: 89)
TYEDFVEGLRV,
(SEQ ID NO: 90)
YEDFVEGLRVF,
(SEQ ID NO: 91)
YEDFVEGLRVFD,
(SEQ ID NO: 92)
YEDFVEGLRVFDK,
(SEQ ID NO: 93)
YEDFVEGLVFDKE,
(SEQ ID NO: 94)
YEDFVEGLVFDKEG,
(SEQ ID NO: 95)
YEDFVEGLVFDKEGN,
(SEQ ID NO: 96)
YEDFVEGLVFDKEGNG,

or a conservatively substituted variant thereof.

In some other embodiments, skeletal muscle myosin chain derived anti-coagulant peptides of the invention has a sequence that is derived from human myosin HC fragment sequence shown in SEQ ID NO:24 (AIFCIQYNIRSFMNVKHWPW). In some of these embodiments, the peptide comprises a sequence that includes the sequence shown in SEQ ID NO:24 or a conservatively substituted variant thereof. For example, the peptide can contain at least 21, 22, 23, 24, 25, 26, 27, 28, 29, 30, 35, 40 or more contiguous amino acid residues of SEQ ID NO:HC encompassing SEQ ID NO:24. In some other embodiments, the peptide comprises a sequence that includes a N-terminally and/or a C-terminal truncation of SEQ ID NO:24 or a conservatively substituted variant thereof. For example, the peptide can contain a variant of SEQ ID NO:24 containing a deletion of 1, 2, 3, 4, 5, 6 or more residues at the N-terminus of SEQ ID NO:24, and/or a deletion of 1, 2, 3, 4, 5, 6 or more residues at the C-terminus of SEQ ID NO:24. Some exemplary peptides in these embodiments include, e.g.,

(SEQ ID NO: 28)
RREAIFCIQYNIRSFMNVKHWPW,
(SEQ ID NO: 29)
EAIFCIQYNIRSFMNVKHWPW,
(SEQ ID NO: 30)
VERREAIFCIQYNIRSFMNVKHWPW,
(SEQ ID NO: 31)
RMVERREAIFCIQYNIRSFMNVKHWPW,
(SEQ ID NO: 32)
YQRMVERREAIFCIQYNIRSFMNVKHWPW,
(SEQ ID NO: 33)
VEYQRMVERREAIFCIQYNIRSFMNVKHWPW,
(SEQ ID NO: 34)
ARVEYQRMVERREAIFCIQYNIRSFMNVKHWPW,
(SEQ ID NO: 35)
ARVEYQRMVERREAIFCIQYNIRSFMNVKHWPW,
(SEQ ID NO: 36)
FLARVEYQRMVERREAIFCIQYNIRSFMNVKHWPW,
(SEQ ID NO: 37)
RGFLARVEYQRMVERREAIFCIQYNIRSFMNVKHWPW,
(SEQ ID NO: 38)
AIFCIQYNIRSFMNVKHWPWMK,
(SEQ ID NO: 39)
AIFCIQYNIRSFMNVKHWPWMKLF,
(SEQ ID NO: 40)
AIFCIQYNIRSFMNVKHWPWMKLFFK,
(SEQ ID NO: 41)
AIFCIQYNIRSFMNVKHWPWMKLFFKIK,
(SEQ ID NO: 42)
AIFCIQYNIRSFMNVKHWPWMKLFFKIKPL,
(SEQ ID NO: 43)
AIFCIQYNIRSFMNVKHWPWMKLFFKIKPLLK,
(SEQ ID NO: 44)
AIFCIQYNIRSFMNVKHWPW MKLFFKIKPLLKSA,
(SEQ ID NO: 45)
AIFCIQYNIRSFMNVKHWPW MKLFFKIKPLLKSAET,

or a conservatively substituted variant thereof.
IV. Variant peptides and derivative compounds

In addition to the peptides noted above, anti-coagulant peptides of the invention also include related or derivative compounds that are derived from the various peptides or polypeptides exemplified herein. These related or derivative compounds encompass variants, analogs, mimetics, complexes or fusion molecules containing the peptides or polypeptides, and other compounds derived from the anti-coagulant myosin peptides or polypeptides. In general, the variant peptides or derivative compounds should possess substantially the same or improved biological activities (e.g., inhibiting myosin supported prothrombin activation) and/or pharmaceutical properties (e.g., stability) as the anti-coagulant peptides exemplified herein (e.g., SEQ ID NOs:10, 21, 23, and 24). Biological activities and pharmaceutical properties of the variants and derivatives of the invention can be readily assessed via methods well known in the art and/or the various assays described herein. These include, e.g., measuring their effect on myosin stimulated thrombin generation using recalcification of human plasma (see Example 6 herein).

In some embodiments, the variant peptides or derivative compounds can include peptides or polypeptides that have substantially identical sequences, e.g., conservatively modified variants, of the exemplified peptides (e.g., SEQ ID NOs:10, 21, 23 and 24). In addition to peptides derived from the exemplified skeletal myosin MYH2, anti-coagulant peptides of the invention also include peptides or polypeptides that are derived from other human skeletal muscle myosin heavy chains. These include, e.g., peptides that are derived from MYH1, MYH3, MYH4 and MYH13 sequences. In some other embodiments, anti-coagulant peptides of the invention can be derived from other human myosins, including cardiac myosins. These include, e.g., peptides or polypeptides that are derived from cardiac myosin sequences (MYH7, MYH7B, MYL2, MYL3). In still some other embodiments, anti-coagulant peptides of the invention also include peptides or polypeptides that are derived from non-human ortholog skeletal myosin sequences. Related or derivative compounds of the invention also encompass variants, analogs, multimers, peptidomimetics, fusions with other molecules (e.g., carrier moieties) or other derivatives that can be generated from the anti-coagulant peptides exemplified herein. These variant peptides and derivative compounds can be subject to the appropriate assays or screening methods to identify anti-coagulation compounds with improved or optimized biological activities (e.g., inhibiting prothrombin activation) and/or pharmaceutical properties (e.g., stability). In some embodiments, the derivative compounds are modified versions of the exemplified peptides which are generated by conservative amino acid substitutions. In some other embodiments, the derivative compounds are variants produced by non-conservative substitutions to the extent that that they substantially retain the activities of those peptides. Modification to an anti-coagulant peptide of the invention can be performed with standard techniques routinely practiced in the art (e.g., U.S. Patent Applications 20080090760 and 20060286636).

In some embodiments, variant peptides or derivative compounds of an anti-coagulant peptide of the invention (e.g., SEQ ID NOs:10, 21, 23 and 24) are analogs or that contain one or more naturally occurring amino acid derivatives of the twenty standard amino acids, for example, 4-hydroxyproline, 5-hydroxylysine, 3-methylhistidine, homoserine, ornithine or carboxyglutamate, and can include amino acids that are not linked by polypeptide bonds. Similarly, they can also be cyclic polypeptides and other conformationally constrained structures. Methods for modifying a polypeptide to generate analogs and derivatives are well known in the art, e.g., Roberts and Vellaccio, The Peptides: Analysis, Synthesis, Biology, Eds. Gross and Meinhofer, Vol. 5, p. 341, Academic Press, Inc., New York, N.Y. (1983); and Burger's Medicinal Chemistry and Drug Discovery, Ed. Manfred E. Wolff, Ch. 15, pp. 619-620, John Wiley & Sons Inc., New York, N.Y. (1995).

In some other embodiments, the derivative compounds of the exemplified anti-coagulant peptides are peptidomimetics. Peptidomimetics based on an anti-coagulant peptide (e.g., SEQ ID NO:10, 21, 23 or 24) substantially retain the activities of the reference peptide. As exemplified herein, they include chemically modified peptides or polypeptides, polypeptide-like molecules containing non-naturally occurring amino acids, peptoids and the like, that have a structure substantially the same as the reference polypeptide or peptide upon which the peptidomimetic is derived (see, for example, Burger's Medicinal Chemistry and Drug Discovery, 1995, supra). For example, the peptidomimetics can have one or more residues chemically derivatized by reaction of a functional side group. In addition to side group derivatizations, a chemical derivative can have one or more backbone modifications including alpha-amino substitutions such as N-methyl, N-ethyl, N-propyl and the like, and alpha-carbonyl substitutions such as thioester, thioamide, guanidino and the like. Typically, a peptidomimetic shows a considerable degree of structural identity when compared to the reference polypeptide and exhibits characteristics which are recognizable or known as being derived from or related to the reference polypeptide. Peptidomimetics include, for example, organic structures which exhibit similar properties such as charge and charge spacing characteristics of the reference polypeptide. Peptidomimetics also can include constrained structures so as to maintain optimal spacing and charge interactions of the amino acid functional groups.

In some embodiments, the variant peptides or derivative compounds of the exemplified anti-coagulant peptides are modified by attaching a specific chemical structure or moiety (staple) to the peptide sequence. A great number of chemical moieties can be used for attachment to the anti-coagulant peptides of the invention, as demonstrated herein (e.g., Examples 8 and 9). As exemplified, some of the chemical modifications to the peptides involve attachment of the chemical structure to one amino acid residue in the peptide sequence. In some other embodiments, the chemical structure (staple) is attached to 2 residues in the peptide sequence. In some embodiments, the attached chemical structure is further conjugated to a lipid or fatty acid to further enhance pharmaceutical or biological activities. In still some other embodiments as exemplified herein, variant peptides with improved properties are realized by an engineered disulfide in the peptide sequence.

In some embodiments, derivative compounds of anti-coagulant peptides described herein are molecules that have the peptides or polypeptides covalently or non-covalently conjugated to a carrier moiety via any conventional methods. A “carrier moiety” (“carrier” or “carrier molecule”) is a conjugation partner capable of enhancing the immunogenicity of a polypeptide. Suitable carriers are typically large, slowly metabolized macromolecules such as proteins; polysaccharides (such as latex functionalized SEPHAROSE™, agarose, cellulose, cellulose beads and the like); polymeric amino acids (such as polyglutamic acid, poly-lysine, and the like); amino acid copolymers; and inactive virus particles or attenuated bacteria, such as Salmonella. In various embodiments, the carrier moiety can be a carrier protein, an immunoglobulin, a Fc domain, a PEG molecule or other polymer. In some embodiments, the carrier moiety is a protein. Integral membrane proteins from, e.g., E. coli and other bacteria are useful conjugation partners. Especially useful carrier proteins are serum albumins, keyhole limpet hemocyanin (KLH), certain immunoglobulin molecules, thyroglobulin, ovalbumin, bovine serum albumin (BSA), tetanus toxoid (TT), and diphtheria toxoid (CRM). In some other embodiments, the carrier moiety can be a polymer other than a protein or polypeptide. Examples of such polymers include, e.g., carbohydrates such as dextran, mannose or mannan.

In some embodiments, the anti-coagulant peptides, variants or derivatives described herein are dimerized or multimerized molecules. In some of these embodiments, dimerization or multimerization of the anti-inflammatory agents can be achieved by covalent attachment to at least one linker moiety. Either homologous multimerization or heterologous multimerization of the anti-coagulant peptides can be achieved via various suitable means. For example, the peptides can be covalently linked via recombinant techniques. In some embodiments, the multimerized molecules of the invention can utilize a carrier moiety noted above. In some embodiments, the peptides or polypeptides can also be conjugated with a C₁₋₁₂ linking moiety optionally terminated with one or two -NH- linkages and optionally substituted at one or more available carbon atoms with a lower alkyl substituent. The anti-coagulant peptides described herein can be joined by other chemical bond linkages, such as linkages by disulfide bonds or by chemical bridges. In some other embodiments, the anti-coagulant peptides described herein can be linked physically in tandem to form a polymer of anti-coagulant peptides. The peptides making up such a polymer can be spaced apart from each other by a peptide linker. In some embodiments, molecular biology techniques well known in the art can be used to create a polymer of peptides. In some embodiments, polyethylene glycol (PEG) may serve as a linker that dimerizes two peptide monomers. For example, a single PEG moiety containing two reactive functional groups may be simultaneously attached to the N-termini of both peptide chains of a peptide dimer. These peptides are referred to herein as “PEGylated peptides.” In some embodiments, the peptide monomers of the invention may be oligomerized using the biotin/streptavidin system.

Methods for stabilizing peptides known in the art may be used with the methods and compositions described herein. As exemplified herein, using D-amino acids, using reduced amide bonds for the peptide backbone, and using non-peptide bonds to link the side chains, including, but not limited to, pyrrolinone and sugar mimetics can each provide stabilization. The design and synthesis of sugar scaffold peptide mimetics are described in the art, e.g., Hirschmann et al., J. Med. Chem. 36, 2441-2448, 1996. Further, pyrrolinone-based peptide mimetics present the peptide pharmacophore on a stable background that has improved bioavailability characteristics. See, e.g., Smith et al., J. Am. Chem. Soc. 122, 11037-11038, 2000.

In some embodiment, derivative compounds of the anti-coagulant peptides include modifications within the sequence, such as, modification by terminal-NH₂ acylation, e.g., acetylation, or thioglycolic acid amidation, by terminal-carboxylamidation, e.g., with ammonia, methylamine, and the like terminal modifications. One can also modify the amino and/or carboxy termini of the polypeptides described herein. Terminal modifications are useful to reduce susceptibility by proteinase digestion, and therefore can serve to prolong half-life of the polypeptides in solution, particularly in biological fluids where proteases may be present. Amino terminus modifications include methylation (e.g., —NHCH₃ or —N(CH₃)₂), acetylation (e.g., with acetic acid or a halogenated derivative thereof such as α-chloroacetic acid, α-bromoacetic acid, or α-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from the group consisting of alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the peptide compound. In some embodiments, the N-terminus is acetylated with acetic acid or acetic anhydride.

As exemplified herein, carboxy terminus modifications include deletion of one or more of the terminal residues, additional or one or more residues (e.g., a poly lysine tail). Carboxy terminus modifications also include replacing the free acid with a carboxamide group or forming a cyclic lactam at the carboxy terminus to introduce structural constraints. One can also cyclize the peptides described herein, or incorporate a desamino or descarboxy residue at the termini of the peptide, so that there is no terminal amino or carboxyl group, to decrease susceptibility to proteases or to restrict the conformation of the peptide. Methods of circular peptide synthesis are known in the art, for example, in U.S. Patent Application No. 20090035814; and Muralidharan and Muir, Nat. Methods, 3:429-38, 2006. C-terminal functional groups of the peptides described herein include amide, amide lower alkyl, amide di(lower alkyl), lower alkoxy, hydroxy, and carboxy, and the lower ester derivatives thereof, and the pharmaceutically acceptable salts thereof. In some embodiments, the variant peptides or derivative compounds of the invention contain a poly-lysine tail at the C-terminus. In various embodiments, the poly-lysine tail can contain from about 3 to about 10 lysine residues.

The anti-coagulant peptide compounds described herein also serve as structural models for non-peptidic compounds with similar biological activity. There are a variety of techniques available for constructing compounds with the same or similar desired biological activity as the exemplified anti-coagulant peptides, but with more favorable activity with respect to solubility, stability, and susceptibility to hydrolysis and proteolysis. See, e.g., Morgan and Gainor, Ann. Rep. Med. Chem. 24:243-252, 1989. These techniques include, but are not limited to, replacing the peptide backbone with a backbone composed of phosphonates, amidates, carbamates, sulfonamides, secondary amines, and N-methylamino acids.

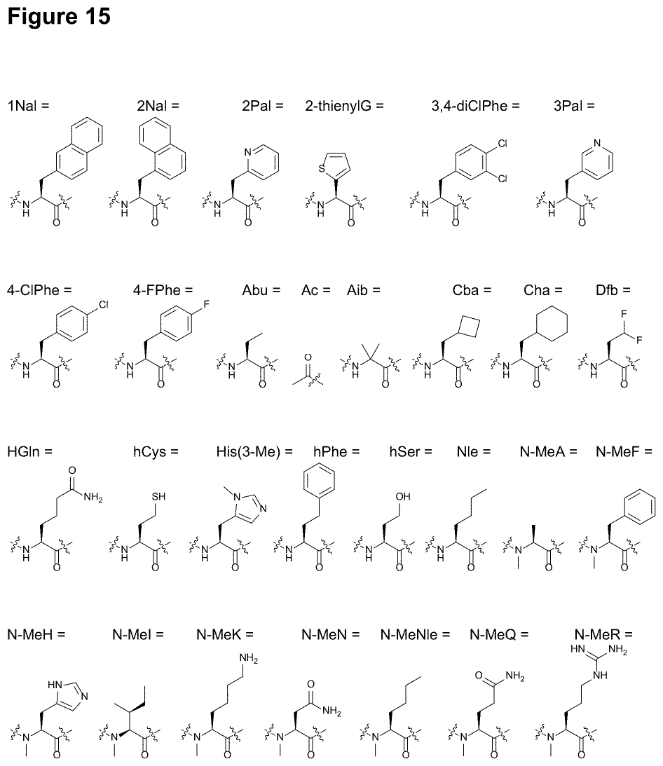

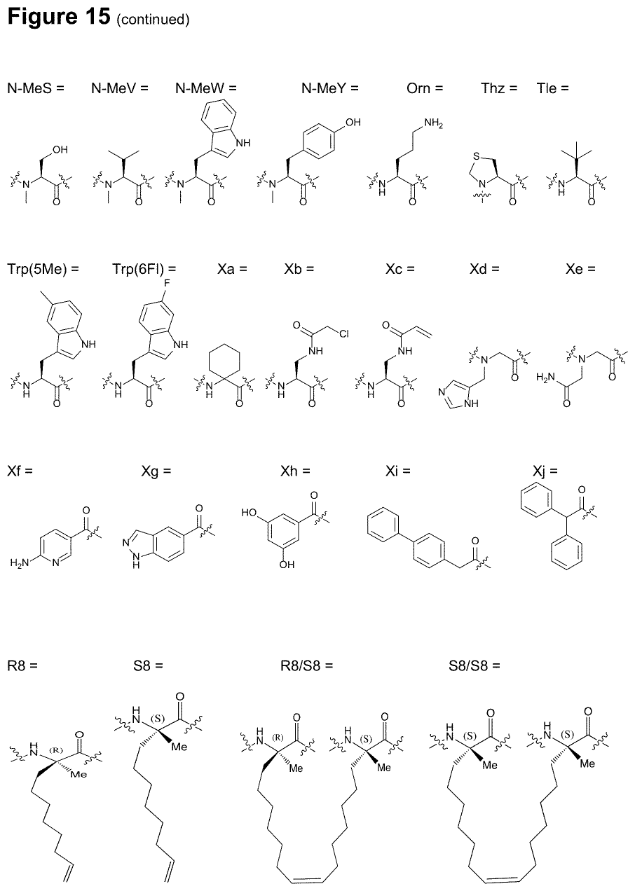

As exemplification, a number of derivative compounds based on HC peptide 796-835 (SEQ ID NO:21; also termed peptide HC061 herein) and residues 816-835 therein (SEQ ID NO:24) with various extent of modifications and optimization were generated and examined for improved activities. Modifications in these peptides, relative to original peptide shown in SEQ ID NO:21, include internal amino acid residue deletions, terminal truncations or additions of amino acid residues (e.g., addition of poly lysine tail), conservative or non-conservative substitutions, substitutions with D-amino acids and non-natural amino acid residue, and attachment of chemical compounds or moieties (“staples”). It was observed that these variants or derivative possess substantively improved biological or pharmaceutical properties, e.g., improved stability and/or enhanced inhibitory activity on myosin mediated prothrombin activation. As shown in the peptide sequences, modifications include substitutions with or additions of well known non-natural amino acids and D-amino acids. Also shown in the sequences are modifications of the myosin derived anti-coagulant peptides with several other amino acid analogs, e.g., 3Pal in peptide HC178. Structures of these amino acid analogs are indicated in FIG. 15. As described in detail in the Examples herein, optimization of the early lead peptides (e.g., Ala scanning) identified important sequence motifs and critical residues that are important to maintain or enhance the anti-coagulant activities of the peptides.

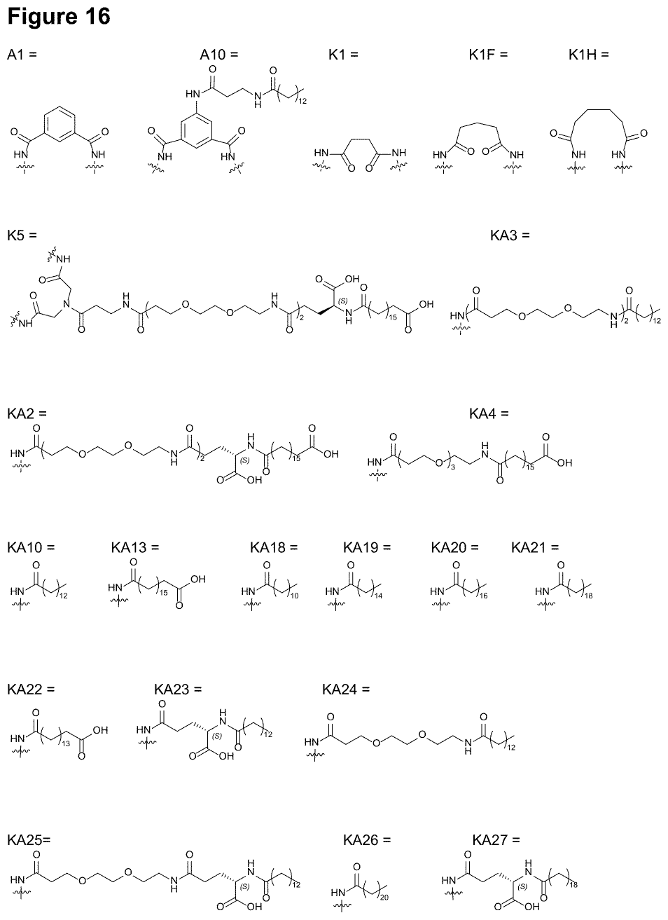

In some embodiments, modifications of the peptides involve attachment, internally or terminally, of various chemical moieties (staples) as indicated under Column “Staple/Lipid” in the table. Structures of some specific staples that were used to modify the myosin-derived anti-coagulant peptides described herein are shown in FIG. 16. Specifically, ‘KA’ staples are single-site lipidation with single attachment point in the peptide sequences. In addition to the staple itself, chemical modifications in these compounds all contain some form of fatty acid moiety attached to the staple. Other than the ‘KA’ staples, all other staple designations in the table refer to chemical moieties that are conjugated to the peptides at two attachment points (forming a cycle). Among these staples, ‘K1’ and ‘A1’ staples do not have any lipid attached to the staple itself. Thus, they are ‘simple stapling and are not ‘trifunctional.’ In contrast, all other non-‘KA’ staples (other than the K1 and A1 staples) have a lipid/fatty acid moiety attached, are ‘trifunctional’. Any of these variant peptides or derivative compounds, as well as similar compounds with substantially identical or conservatively modified sequences, are all within the scope of the invention. Examples of lipid moieties that can be linked or conjugated to the staples are described in the Examples herein as well as PCT publications WO2015/095406 and WO2016/205488.

In some exemplified embodiments, the derivative compounds contain deletion of one or more terminal or internal residues of SEQ ID NO:24. In some other embodiments, the exemplified compounds derived from SEQ ID NO:21 or 24 contain one or more amino acid substitutions in SEQ ID NO:24 with non-natural amino acid residues, amino acid analogs or D-amino acid residues. For example, some of these compounds contain SEQ ID NO:24 with one or more of the following substitutions in SEQ ID NO:24: Cys⁴→Lys, Thr or Ser, Ser¹¹→Lys, Met¹³→Nle, and Asn¹⁴→N-MeN. Some other exemplified compounds derived from SEQ ID NO:21 or 24 contain SEQ ID NO:24 with C-terminal additions of (1) an Nle residue and (2) a poly-lysine tail that consists of about 4 to about 8 lysine residues. In some other embodiments, the derivative compounds contain SEQ ID NO:24 that is modified by conjugating a chemical moiety (staple) to one or two residues in the sequence of the peptide. In some of these embodiments, the chemical moiety is attached to an engineered Lys residue that substitutes for Ser¹¹ in SEQ ID NO:24. In some of the embodiments, the attached chemical moiety is KA21, KA10, KA26, KA18, KA19, KA3, KA24, KA23, KA27 or KA22 as shown in FIG. 16.

The various myosin derived anti-coagulant peptides described herein, including variants and derivatives thereof, can be chemically synthesized and purified by standard chemical or biochemical methods that are well known in the art. These include standard solid-phase peptide synthesis (SPPS) techniques and purification via HPLC as exemplified herein. See additionally, e.g., Merrifield, J. Am. Chem. Soc. 85:2149-2154, 1963; “Peptide synthesis and applications” in Methods in molecular biology Vol. 298, Ed. by John Howl; “Chemistry of Peptide Synthesis” by N. Leo Benoiton, 2005, CRC Press, (ISBN-13: 978-1574444544); and “Chemical Approaches to the Synthesis of Peptides and Proteins” by P. Lloyd-Williams, et. al., 1997, CRC-Press, (ISBN-13: 978-0849391422), Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906); U.S. Pat. Nos. 4,965,343, and 5,849,954.

In some embodiments, the peptides can be obtained via solid phase synthesis using peptide synthesizing machines. Commercial peptide synthesizing machines are available for solid phase peptide synthesis. For example, the Advanced Chemtech Model 396 Multiple Peptide Synthesizer and an Applied Biosystems Model 432A Peptide synthesizer are suitable. There are commercial companies that make custom synthetic peptides to order, e.g., Abbiotec, Abgent, AnaSpec Global Peptide Services, LLC., Invitrogen, and rPeptide, LLC.

### V. Polynucleotides and Expression Constructs

The invention also provides isolated or substantially purified polynucleotides (DNA or RNA) which encode the various myosin derived anti-coagulant peptides or polypeptides (e.g., heterologous or homologous fusions of the peptides shown in SEQ ID NOs:1-24), as well as the variants or derivative sequences described herein. Expression vectors and engineered host cells harboring the vectors for expressing polynucleotides encoding the peptides or polypeptides are also provided in the invention. The polynucleotide encoding the peptides or polypeptides are operationally linked to a promoter in the expression vectors. The expression construct can further comprise a secretory sequence to assist purification of the peptide from the cell culture medium. The host cells to which the vectors are introduced can be any of a variety of expression host cells well known in the art, e.g., bacteria (e.g., E. coli), yeast cell, or mammalian cells.

The polynucleotides and related vectors can be readily generated with standard molecular biology techniques or the protocols exemplified herein. For example, general protocols for cloning, transfecting, transient gene expression and obtaining stable transfected cell lines are described in the art, e.g., Sambrook et al., Molecular Cloning: A Laboratory Manual, Cold Spring Harbor Press, N.Y., (3^{rd} ed., 2000); and Brent et al., Current Protocols in Molecular Biology, John Wiley & Sons, Inc. (ringbou ed., 2003). Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

The selection of a particular vector depends upon the intended use of the fusion polypeptides. For example, the selected vector must be capable of driving expression of the fusion polypeptide in the desired cell type, whether that cell type be prokaryotic or eukaryotic. Many vectors contain sequences allowing both prokaryotic vector replication and eukaryotic expression of operably linked gene sequences. Vectors useful for the invention may be autonomously replicating, that is, the vector exists extrachromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors and in stably transfected cell lines. Both viral-based and nonviral expression vectors can be used to produce the immunogens in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). Useful viral vectors include vectors based on lentiviruses or other retroviruses, adenoviruses, adenoassociated viruses, Cytomegalovirus, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

Depending on the specific vector used for expressing the fusion polypeptide, various known cells or cell lines can be employed in the practice of the invention. The host cell can be any cell into which recombinant vectors carrying a fusion of the invention may be introduced and wherein the vectors are permitted to drive the expression of the fusion polypeptide is useful for the invention. It may be prokaryotic, such as any of a number of bacterial strains, or may be eukaryotic, such as yeast or other fungal cells, insect or amphibian cells, or mammalian cells including, for example, rodent, simian or human cells. Cells expressing the fusion polypeptides of the invention may be primary cultured cells or may be an established cell line. Thus, in addition to the cell lines exemplified herein (e.g., CHO cells), a number of other host cell lines capable well known in the art may also be used in the practice of the invention. These include, e.g., various Cos cell lines, HeLa cells, HEK293, AtT20, BV2, and N18 cells, myeloma cell lines, transformed B-cells and hybridomas.

The use of mammalian tissue cell culture to express polypeptides is discussed generally in, e.g., Winnacker, From Genes to Clones, VCH Publishers, N.Y., N.Y., 1987. The fusion polypeptide-expressing vectors may be introduced to the selected host cells by any of a number of suitable methods known to those skilled in the art. For the introduction of fusion polypeptide-encoding vectors to mammalian cells, the method used will depend upon the form of the vector. For plasmid vectors, DNA encoding the fusion polypeptide sequences may be introduced by any of a number of transfection methods, including, for example, lipid-mediated transfection (“lipofection”), DEAE-dextran-mediated transfection, electroporation or calcium phosphate precipitation. These methods are detailed, for example, in Brent et al., supra. Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, Life Technologies, JBL Scientific, MBI Fermentas, PanVera, Promega, Quantum Biotechnologies, Sigma-Aldrich, and Wako Chemicals USA.

For long-term, high-yield production of recombinant fusion polypeptides, stable expression is preferred. Rather than using expression vectors which contain viral origins of replication, host cells can be transformed with the fusion polypeptide-encoding sequences controlled by appropriate expression control elements (e.g., promoter, enhancer, sequences, transcription terminators, polyadenylation sites, etc.), and selectable markers. The selectable marker in the recombinant vector confers resistance to the selection and allows cells to stably integrate the vector into their chromosomes. Commonly used selectable markers include neo, which confers resistance to the aminoglycoside G-418 (Colberre-Garapin, et al., J. Mol. Biol., 150:1, 1981); and hygro, which confers resistance to hygromycin (Santerre et al., Gene, 30: 147, 1984). Through appropriate selections, the transfected cells can contain integrated copies of the fusion polypeptide encoding sequence.

VI. Antibodies Targeting Myosin Derived Anti-Coagulant Peptides

The invention additionally provides antibodies and related antigen-binding fragments that can specifically recognize one of the myosin derived anti-coagulant peptides or derivative compounds described herein. Unless otherwise noted, antibodies or antigen-binding molecules of the invention can have sequences derived from any vertebrate, camelid, avian or pisces species. They can be generated using any suitable technology, e.g., hybridoma technology, ribosome display, phage display, gene shuffling libraries, semi-synthetic or fully synthetic libraries or combinations thereof. As detailed herein, antibodies or antigen-binding molecules of the invention include intact antibodies, antigen-binding polypeptide chains and other designer antibodies (see, e.g., Serafini, J. Nucl. Med. 34:533-6, 1993).

Antibodies or antigen-binding molecules suitable for the invention also include antibody fragments which contain the antigen-binding portions of an intact antibody that retain capacity to bind the cognate antigen (e.g., peptides shown in SEQ ID NOs:10, 21, 23, and 24), including the specific myosin epitope that is present on one of the anti-coagulant peptides or derivatives described herein. Examples of such antibody fragments include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546, 1989), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al., Science 242:423-426, 1988; and Huston et al., Proc. Natl. Acad. Sci. USA 85:5879-5883, 1988.

Antibodies or antigen-binding molecules that specifically target the myosin derived anti-coagulant peptides of the invention further include one or more immunoglobulin chains that are chemically conjugated to, or expressed as, fusion proteins with other proteins. It also includes bispecific antibody. A bispecific or bifunctional antibody is an artificial hybrid antibody having two different heavy/light chain pairs and two different binding sites. Other antigen-binding fragments or antibody portions of the invention include bivalent scFv (diabody), bispecific scFv antibodies where the antibody molecule recognizes two different epitopes, single binding domains (dAbs), and minibodies.

Polyclonal and monoclonal antibodies that specifically target a myosin derived anti-coagulant peptides or derivative compounds described herein can be readily produced with routinely practiced immunology methods, e.g., hybridoma technologies. See, e.g., Harlow & Lane, Using Antibodies, A Laboratory Manual, Cold Spring Harbor Laboratory Press, Cold Spring Harbor, N.Y., 1998. To enhance immunogenicity for antibody productions, the myosin-derived peptide can be conjugated to a large carrier molecule, e.g., a carrier protein like KLH as exemplified herein. Other than intact antibodies, the various other forms of antibodies or antigen-binding fragments can be produced by enzymatic or chemical modification of the intact antibodies, or synthesized de novo using recombinant DNA methodologies (e.g., single chain Fv), or identified using phage display libraries. Methods for generating these antibodies or antigen-binding molecules are all well known in the art. For example, single chain antibodies can be identified using phage display libraries or ribosome display libraries, gene shuffled libraries (see, e.g., McCafferty et al., Nature 348:552-554, 1990; and U.S. Pat. No. 4,946,778). In particular, scFv antibodies can be obtained using methods described in, e.g., Bird et al., Science 242:423-426, 1988; and Huston et al., Proc. Natl. Acad. Sci. USA 85:5879-5883, 1988. Fv antibody fragments can be generated as described in Skerra and PlUckthun, Science 240:1038-41, 1988. Disulfide-stabilized Fv fragments (dsFvs) can be made using methods described in, e.g., Reiter et al., Int. J. Cancer 67:113-23, 1996. Similarly, single domain antibodies (dAbs) can be produced by a variety of methods described in, e.g., Ward et al., Nature 341:544-546, 1989; and Cai and Garen, Proc. Natl. Acad. Sci. USA 93:6280-85, 1996. Camelid single domain antibodies can be produced using methods well known in the art, e.g., Dumoulin et al., Nature Struct. Biol. 11:500-515, 2002; Ghahroudi et al., FEBS Letters 414:521-526, 1997; and Bond et al., J Mol Biol. 332:643-55, 2003.

### VII. Therapeutic Applications and Pharmaceutical Compositions

The invention provides methods for preventing and/or treating thrombosis by specifically targeting myosin-mediated prothrombin activation with the anti-coagulant peptides, variants or derivatives described herein. Prothrombin activations mediated different types of myosin are all suitable for treatment with methods of the invention. For example, the anti-coagulant activities of the compounds described herein can be employed for inhibiting thrombosis that is supported by either skeletal myosin or cardiac myosin, as exemplified herein. Preferably, therapeutic methods of the invention are directed to treating mammalian subjects. In some of these embodiments, the subject is a human patient. The methods can be readily employed in various clinical settings, e.g., to treat or prevent thrombosis in acute trauma patients. Particularly suitable for the therapeutic methods of the invention are post-trauma patients having or at risk of developing acute trauma coagulopathy. In some other embodiments, the subject to be treated with methods of the inventon is one who is afflicted with or at risk of having coronary thrombosis.

Typically, the anti-coagulant peptides and derivative compounds of the invention (e.g., peptides of SEQ ID NOs:10, 21, 23 and 24 and compounds shown in Table 2) can be formulated in pharmaceutical compositions for the therapeutic or prophylactic applications disclosed herein. Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. The pharmaceutical compositions of the invention may be administered by any known route. By way of example, the composition may be administered by a mucosal, pulmonary, topical, or other localized or systemic route (e.g., enteral and parenteral). Parenteral administration includes subcutaneous, intradermal, intramuscular, intravenous, intra-arterial, intrathecal, and other injection or infusion techniques, without limitation.

Suitable choices in amounts and timing of doses, formulation, and routes of administration can be made with the goals of achieving a favorable response in the subject (i.e., efficacy or therapeutic), and avoiding undue toxicity or other harm thereto (i.e., safety). Administration may be by bolus or by continuous infusion. Bolus refers to administration of a drug (e.g., by injection) in a defined quantity (called a bolus) over a period of time. Continuous infusion refers to continuing substantially uninterrupted the introduction of a solution into a blood vessel for a specified period of time. A bolus of the formulation administered only once to a subject is a convenient dosing schedule, although in the case of achieving an effective concentration of the anti-coagulant peptides and derivative compound in the brain more frequent administration may be required. Treatment may involve a continuous infusion (e.g., for 3 hr after stroke) or a slow infusion (e.g., for 24 hr to 72 hr when given within 6 hr of stroke). Alternatively, it may be administered every other day, once a week, or once a month. Dosage levels of active ingredients in a pharmaceutical composition can also be varied so as to achieve a transient or sustained concentration of the compound or derivative thereof in a subject and to result in the desired therapeutic response.

The pharmaceutical compositions of the invention may be administered as a formulation, which is adapted for direct application to the central nervous system, or suitable for passage through the gut or blood circulation. Alternatively, pharmaceutical compositions may be added to the culture medium. In addition to active compound, such compositions may contain pharmaceutically acceptable carriers and other ingredients known to facilitate administration and/or enhance uptake. It may be administered in a single dose or in multiple doses, which are administered at different times. A unit dose of the composition is an amount of the anti-coagulant peptides and derivative compounds which provides effective inhibition of undesired thrombosis. Measurement of such values can be performed with standard techniques well known in the art, e.g., clinical laboratories routinely determine these values with standard assays and hematologists classify them as normal or abnormal depending on the situation.

When administered to a subject in vivo, the pharmaceutical compositions typically contain a therapeutically effective amount of the anti-coagulant peptides and derivative compound. A therapeutically effective amount is the total amount of the anti-coagulant peptides and derivative compound that achieves the desired thrombosis-inhibiting effect. Depending on the species of the subject or disease to be treated, the therapeutic amount may be about 0.01 mg/kg/hr to about 1.1 mg/kg/hr if administered by continuous infusion over 4 hour to 96 hour, to as little as about 0.01 mg/kg/hr to about 0.10 mg/kg/hr for about 24 hours. Preferably, the therapeutic dose would be administered by continuous infusion for about 4 to about 72 hours. More preferably, by continuous infusion for about 4 to about 48 hours. More preferably, by continuous infusion for about 12 to about 48 hours. More preferably, by continuous infusion for about 12 to about 36 hours. More preferably, by continuous infusion for about 4 to about 36 hours. More preferably, by continuous infusion for about 12 to about 24 hours. Most preferably, by continuous infusion for about 24 hours. In other examples, a therapeutically effective amount for bolus administration can typically be 2 mg/kg or less, 1 mg/kg or less, 0.5 mg/kg or less, 0.04 mg/kg or less, 0.03 mg/kg or less, 0.02 mg/kg or less, 0.01 mg/kg or less, or 0.005 mg/kg or less. Typically, the therapeutic amount may be based on titering to a blood level amount of the anti-coagulant peptides and derivative compound of about 0.01 μg/ml to about 1.6 μg/ml, preferably from about 0.01 μg/ml to about 0.5 μg/ml. It is also within the skill of the art to start doses at levels lower than required to achieve the desired therapeutic effect and to gradually increase the dosage until the desired effect is achieved. It is likewise within the skill of the art to determine optimal concentrations of variants to achieve the desired effects in the in vitro and ex vivo preparations of the invention. Depending on initial assay results, optimal concentrations can be in the range of, e.g., about 1-1,000 nM or about 1-200 μM depending on the general nature of the compound.

Human MYH2 sequence (NP_060004)
(SEQ ID NO: 25)
MSSDSELAVF GEAAPFLRKS ERERIEAQNR PFDAKTSVFV
AEPKESFVKG TIQSREGGKV TVKTEGGATL TVKDDQVFPM
NPPKYDKIED MAMMTHLHEP AVLYNLKERY AAWMIYTYSG
LFCVTVNPYK WLPVYKPEVV TAYRGKKRQE APPHIFSISD
NAYQFMLTDR ENQSILITGE SGAGKTVNTK RVIQYFATIA
VTGEKKKEEI TSGKIQGTLE DQIISANPLL EAFGNAKTVR
NDNSSRFGKF IRIHFGTTGK LASADIETYL LEKSRVVFQL
KAERSYHIFY QITSNKKPEL IEMLLITTNP YDYPFVSQGE
ISVASIDDQE ELMATDSAID ILGFTNEEKV SIYKLTGAVM
HYGNLKFKQK QREEQAEPDG TEVADKAAYL QSLNSADLLK
ALCYPRVKVG NEYVTKGQTV EQVSNAVGAL AKAVYEKMFL
WMVARINQQL DTKQPRQYFI GVLDIAGFEI FDFNSLEQLC
INFTNEKLQQ FFNHHMFVLE QEEYKKEGIE WTFIDFGMDL
AACIELIEKP MGIFSILEEE CMFPKATDTS FKNKLYDQHL
GKSANFQKPK VVKGKAEAHF ALIHYAGVVD YNITGWLEKN
KDPLNETVVG LYQKSAMKTL AQLFSGAQTA EGEGAGGGAK
KGGKKKGSSF QTVSALFREN LNKLMTNLRS THPHFVRCII
PNETKTPGAM EHELVLHQLR CNGVLEGIRI
CRKGFPSRILYADFKQRYKV LNASAIPEGQ FIDSKKASEK
LLASIDIDHT QYKFGHTKVF FKAGLLGLLE EMRDDKLAQL
ITRTQARCRG FLARVEYQRM VERREAIFCI QYNIRSFMNV
KHWPWMKLFF KIKPLLKSAE TEKEMATMKE EFQKIKDELA
KSEAKRKELE EKMVTLLKEK NDLQLQVQAE AEGLADAEER
CDQLIKTKIQ LEAKIKEVTE RAEDEEEINA ELTAKKRKLE
DECSELKKDI DDLELTLAKV EKEKHATENK VKNLTEEMAG
LDETIAKLTK EKKALQEAHQ QTLDDLQAEE DKVNTLTKAK
IKLEQQVDDL EGSLEQEKKL RMDLERAKRK LEGDLKLAQE
SIMDIENEKQ QLDEKLKKKE FEISNLQSKI EDEQALGIQL
QKKIKELQAR IEELEEEIEA ERASRAKAEK QRSDLSRELE
EISERLEEAG GATSAQIEMN KKREAEFQKM RRDLEEATLQ
HEATAATLRK KHADSVAELG EQIDNLQRVK QKLEKEKSEM
KMEIDDLASN VETVSKAKGN LEKMCRTLED QLSELKSKEE
EQQRLINDLT AQRGRLQTES GEFSRQLDEK EALVSQLSRG
KQAFTQQIEE LKRQLEEEIK AKNALAHALQ SSRHDCDLLR
EQYEEEQESK AELQRALSKA NTEVAQWRTK YETDAIQRTE
ELEEAKKKLA QRLQAAEEHV EAVNAKCASL EKTKQRLQNE
VEDLMLDVER TNAACAALDK KQRNFDKILA EWKQKCEETH
AELEASQKEA RSLGTELFKI KNAYEESLDQ LETLKRENKN
LQQEISDLTE QIAEGGKRIH ELEKIKKQVE QEKCELQAAL
EEAEASLEHE EGKILRIQLE LNQVKSEVDR KIAEKDEEID
QLKRNHIRIV ESMQSTLDAE IRSRNDAIRL KKKMEGDLNE
MEIQLNHANR MAAEALRNYR NTQGILKDTQ IHLDDALRSQ
EDLKEQLAMV ERRANLLQAE IEELRATLEQ TERSRKIAEQ
ELLDASERVQ LLHTQNTSLI NTKKKLETDI SQMQGEMEDI
LQEARNAEEK AKKAITDAAM MAEELKKEQD TSAHLERMKK
NMEQTVKDLQ LRLDEAEQLA LKGGKKQIQK LEARVRELEG
EVESEQKRNA EAVKGLRKHE RRVKELTYQT EEDRKNILRL
QDLVDKLQAK VKSYKRQAEE AEEQSNTNLA KFRKLQHELE
EAEERADIAE SQVNKLRVKS REVHTKVISE E
Human MYL1 sequence (NP_524144)
(SEQ ID NO: 26)
MAPKKDVKKP VAAAAAAPAP APAPAPAPAP AKPKEEKIDL
SAIKIEFSKE QQDEFKEAFL LFDRTGDSKI TLSQVGDVLR
ALGTNPTNAE VRKVLGNPSN EELNAKKIEF EQFLPMMQAI
SNNKDQATYE DFVEGLRVFD KEGNGTVMGA ELRHVLATLG
EKMKEEEVEA LMAGQEDSNG CINYEAFVKH IMSI
Human MYLPF sequence (NP_037424)
(SEQ ID NO: 27)
MAPKRAKRRT VEGGSSSVFS MFDQTQIQEF KEAFTVIDQN
RDGIIDKEDL RDTFAAMGRL NVKNEELDAM MKEASGPINF
TVFLTMFGEK LKGADPEDVI TGAFKVLDPE GKGTIKKKFL
EELLTTQCDR FSQEEIKNMW AAFPPDVGGN VDYKNICYVI
THGDAKDQE

### EXAMPLES

The following examples are provided to further illustrate the invention but not to limit its scope. Other variants of the invention will be readily apparent to one of ordinary skill in the art and are encompassed by the appended claims.

### Example 1

### Identification of Anticoagulant Myosin ELC Peptides

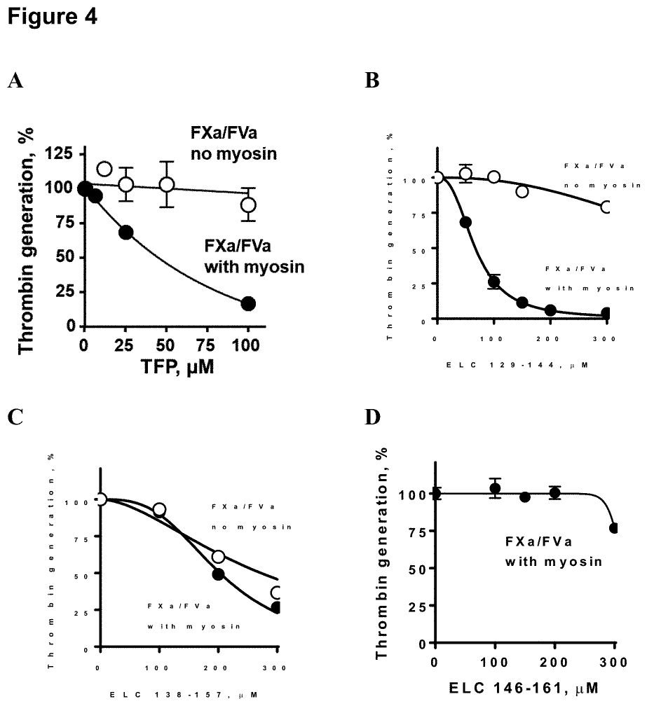

TFP, an allosteric effector for myosin motor activity, inhibits myosin-supported prothrombin activation, as previously reported (1) and as confirmed in dose-dependent studies here (FIG. 4A). However, several other allosteric myosin effectors, namely (-) blebbistatin (up to 65 μM), omecamtiv mecarbil (CK-1827452) (up to 50 μM), and N-benzyl-p-toluene sulfonamide (BTS)(up to 0.7 mM), did not inhibit myosin's procoagulant activity (data not shown). This led us to hypothesize that myosin's TFP binding region on the ELC in the neck region directly contributes to myosin's procoagulant activity. Thus, 3 overlapping 16-mer to 20-mer peptides with ELC sequences (Table 1) were synthesized and tested for inhibition of myosin-enhanced or phospholipid-enhanced prothrombin activation by purified factors Xa and Va in the presence of Ca⁺⁺ ions. The combination of these 3 prothrombin-activating factors (Xa, Va and Ca⁺⁺ ions) is termed the prothrombinase complex while activation of prothrombin to generate alpha-thrombin is termed prothrombinase activity. Two peptides, namely ELC129-144 and ELC138-157, inhibited the myosin-supported prothrombin activation (FIG. 4). Peptide ELC138-157 inhibited prothrombin activation even in the absence of myosin, whereas ELC129-144 did not (FIG. 4). This suggests that peptide ELC129-144 specifically inhibited myosin-supported prothrombin activation, rather than inhibiting myosin-independent clotting factor interactions. This encouraged us to extend the screening for potentially inhibitory peptides to 19 additional peptides containing sequences found on the 3 myosin polypeptides that are clustered in the neck region.

TABLE 1
Initial ELC Peptides screened for inhibition
of myosin-enhanced prothrombin activation.

| residues | Sequence (SEQ ID NO:) |
|---|---|
| ELC 129-144 | Ac-YEDFVEGLRVFDKEGN-NH2 (1) |
| ELC 138-157 | Ac-RVFDKEGNGTVMGAELRHVL-NH2 (2) |
| ELC 146-161 | Ac-GTVMGAELRHVLATLGEK-NH2 (3) |

### Example 2

### Screening for Anticoagulant Peptides Representing Myosin's Neck Region

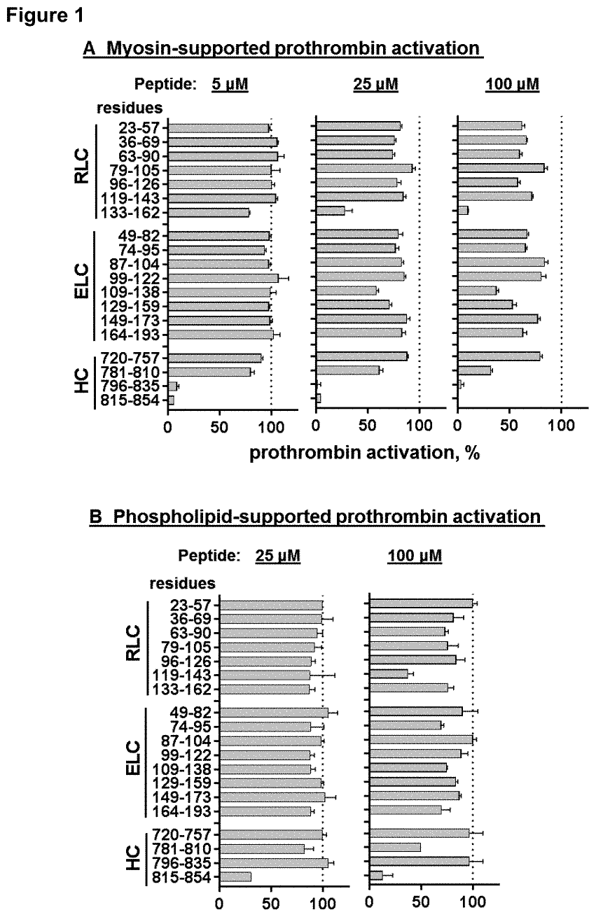

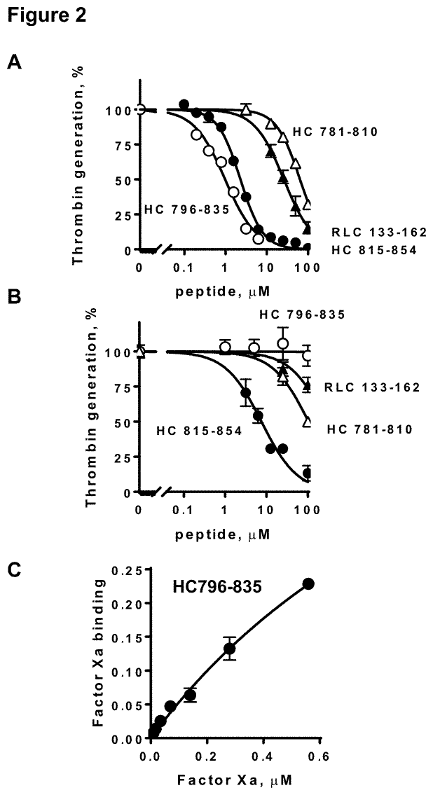

Nineteen peptides from skeletal muscle myosin's neck region, 4 HC peptides (MYH2 sequences), 8 ELC peptides (MYL1 sequences) and 7 RLC peptides (MYLPF sequences) (Table 2) were synthesized and tested at 3 different concentrations for their inhibition of myosin-supported prothrombin activation by purified factor Xa, factor Va, and Ca⁺⁺ ions (FIG. 1A). Peptides ELC109-138 and ELC129-159 inhibited myosin-supported prothrombin activation at 100 μM, whereas their partially overlapping neighbor peptides ELC99-122 and ELC149-173 did not. Three HC peptides (peptides HC781-810, HC796-835, HC815-854) and one RLC peptide (RLC133-162) inhibited myosin-supported prothrombin activation at 100 μM, and each was also inhibitory, to varying degrees, when assayed at 5 μM. Dose-dependency inhibition assays gave IC50 values for the peptides HC781-810, HC796-835, HC815-854, and RLC133-162 of 64, 1.2, 2.3 and 26 μM, respectively (FIG. 2A).

TABLE 2
Amino acid sequences of peptides representing human
skeletal muscle myosin neck region.

| | residues | Sequence (SEQ ID NO:) |
|---|---|---|
| RLC | 23-57 | Ac-DQTQIQEFKEAFTVIDQNRDGIIDKEDLRDTFAAM-NH2 (4) |
| | 36-69 | Ac-VIDQNRDGIIDKEDLRDTFAAMGRLNVKNEELDA-NH2 (5) |
| | 63-90 | Ac-KNEELDAMMKEASGPINFTVFLTMFGEK-NH2 (6) |
| | 79-105 | Ac-NFTVFLTMFGEKLKGADPEDVITGAFK-NH2 (7) |
| | 96-126 | Ac-PEDVITGAFKVLDPEGKGTIKKKFLEELLTT-NH2 (8) |
| | 119-143 | Ac-FLEELLTTQCDRFSQEEIKNMWAAF-NH2 (9) |
| | 133-162 | Ac-QEEIKNMWAAFPPDVGGNVDYKNICYVITH-NH2 (10) |
| ELC | 49-82 | Ac-KEQQDEFKEAFLLFDRTGDSKITLSQVGDVLRAL-NH2 (11) |
| | 74-95 | Ac-QVGDVLRALGTNPTNAEVRKVL-NH2 (12) |
| | 87-104 | Ac-TNAEVRKVLGNPSNEELN-NH2 (13) |
| | 99-122 | Ac-SNEELNAKKIEFEQFLPMMQAISN-NH2 (14) |
| | 109-138 | Ac-EFEQFLPMMQAISNNKDQATYEDFVEGLRV-NH2 (15) |
| | 129-159 | Ac-YEDFVEGLRVFDKEGNGTVMGAELRHVLATL-NH2 (16) |
| | 149-173 | Ac-GAELRHVLATLGEKMKEEEVEALMA-NH (17) |
| | 164-193 | Ac-KEEEVEALMAGQEDSNGCINYEAFVKHIMS-NH2 (18) |
| HC | 720-757 | LYADFKQRYKVLNASAIPEGQFIDSKKASEKLLASIDI-NH2 (19) |
| | 781-810 | EMRDDKLAQLITRTQARCRGFLARVEYQRM-NH2 (20) |
| | 796-835 | ARCRGFLARVEYQRMVERREAIFCIQYNIRSFMNVKHWPW-NH2 (21) |
| | 815-854 | EAIFCIQYNIRSFMNVKHWPWMKLFFKIKPLLKSAETEKE-NH2 (22) |

HC, ELC and RLC denote skeletal muscle myosin heavy chain, essential light chain and regulatory light chain, respectively. Amino acid residue numbers for HC, ELC and RLC correspond to human MYH2, MYL1 and MYLPL. Ac denotes acetylation.

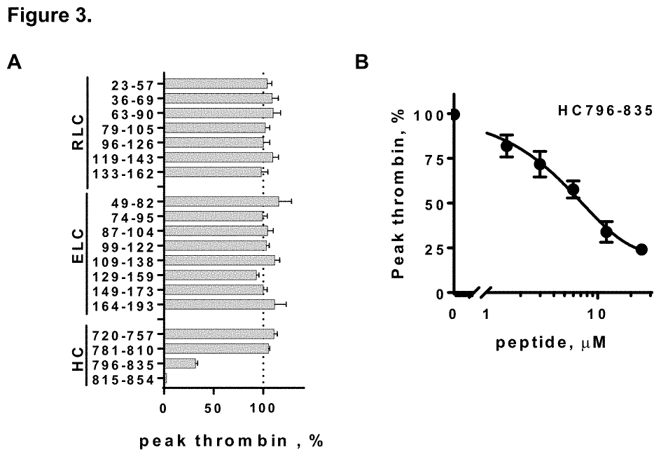

Peptides HC781-810 and HC815-854 inhibited phospholipid vesicles containing 20% phosphatidylserine (PS)-supported prothrombin activation (IC₅₀=7.5 and 104 μM, respectively) (FIGS. 2B and 3B). In contrast, the inhibitory effects of peptides HC796-835, RLC133-162, ELC109-138 and ELC129-159 on phospholipid-supported prothrombin activation were not apparent when compared with their strong inhibitory effects on myosin-supported prothrombin activation (FIGS. 2, 3A and 3B). This suggests that peptides HC796-835, RLC133-162, ELC109-138 and ELC129-159 specifically inhibited myosin-supported prothrombin activation.

### Example 3

### Anticoagulant Effects of Myosin Peptides on Thrombin Generation in Plasma

The 19 synthetic peptides (Table 2) representing the myosin neck region were screened at 25 μM (final concentration) for their inhibition of Ca'-induced thrombin generation in human plasma which contains circulating levels of myosin. Among the 19 peptides, HC796-835 and HC815-854 significantly inhibited thrombin generation when screened at 25 μM in plasma. (FIG. 3A).

### Example 4

### Factor Xa Binding to Peptide

To test the hypothesis that the inhibitory myosin peptides bind factor Xa, various concentrations of factor Xa were incubated in microtiter plate wells that had been coated with various peptides for binding studies. Immobilized peptide HC796 bound factor Xa, and analysis of dose-dependent binding assays were fit with a single binding curve indicating that factor Xa was bound with an apparent Kd of 1.4 μM (FIG. 2C). When the ability of HC796-835 to inhibit thrombin generation in plasma assays was assayed, it was an excellent anticoagulant with IC₅₀ of ˜5.5 μM (FIG. 3B). Since plasma contains circulating levels of myosin whose procoagulant activity is inhibited by anti-myosin antibodies (1), this observation indicates that HC796-835 inhibits myosin's procoagulant activity in the plasma milieu.

### Example 5

### Some Exemplified Materials and Methods

Materials: Human factors Xa and Va were from Hematologic Technologies Inc. (Essex Junction, Vt.), prothrombin from Enzyme Research Laboratories (South Bend, Ind.), thrombin chromogenic substrate (H-D-Phe-Pip-Arg-pNA. 2HCl) from Molecular Innovations, Inc. (Novi Mich.), fluorogenic substrate I-1140 from Bachem Bioscience Inc. (King of Prussia, Pa.), Innovin from DADE Behring, Marburg, Germany, rabbit skeletal muscle myosin from Cytoskeleton, Inc. (Denver, Colo.) or Sigm, fatty acid-free and protease-free bovine serum albumin (BSA) from Sigma, trifluoperazine (TFP), (−)-blebbistatin, CK-1827452, and BTS from MP biochemical (Santa Ana, CA), Cayman Chemical Company (Ann Arbor, Michigan), Toronto Research Chemicals Inc (North York, Ontario, Canada), and MilliporeSigma (Burlington, Mass.), respectively. L-α-PS and L-α-PC (PC)(each from porcine brain) were from Avanti Polar Lipids (Alabaster, Ala.).

Peptide design and synthesis: 3 peptides representing 129-161 amino acid residues of skeletal muscle myosin ELC and containing overlaps of 7-10 amino acids were synthesized using standard solid phase peptide synthesis and Fmoc chemistry by Synthetic Biomolecules (San Diego, Calif.) and purified to >90% by HPLC (Table 1).

Nineteen 18-40-mer peptides containing 6-22 amino acid-overlaps representing the skeletal muscle myosin neck region were synthesized using standard solid phase peptide synthesis and Fmoc chemistry by Cellmano Biotech Limited (Hefei, China), Shanghai Apeptide Co (Shanghai, China) or Shanghai Dechi Biosciences Co. (Shanghai, China), and purified to >90% by HPLC. Peptide identity was confirmed by high resolution mass spectra (HRMS) that were recorded on an Agilent 1200 Series Accurate Mass Time-of-Flight (TOF) with an Aeris Widepore column (XB-C8, 3.6 μm particle size, 150×2.1 mm, flow: 0.5 mL/min).

Activation of prothrombin by prothrombinase complex: Skeletal muscle myosin (2 nM final) was incubated with factor Va (5 nM, final) and factor Xa (0.2 nM, final) with 5 mM CaCl₂ in Tris buffered saline, pH7.4 (TB S) containing 0.5% albumin (TBSA) in the presence or absence of synthetic peptides for 10 min at room temperature (1). PC/PS (80%:20%) vesicles (4 μM, final) were incubated with factor Va (5 nM, final) and factor Xa (0.2 nM, final) with 5 mM CaCl₂ in TB SA in the presence or absence of synthetic peptides for 20, 40 and 60 seconds at room temperature (1). Thrombin generation was initiated by the addition of prothrombin (0.75 μM, final, unless noted otherwise). The reaction was quenched by 10 mM EDTA, and the rate of thrombin formation was quantified by measuring thrombin concentration as the rate of thrombin substrate hydrolysis. Prothrombin (0.75 μM, final) activation in the absence of factor Va was also determined using factor Xa alone. Prothrombin was mixed with various concentrations of peptides at room temperature and then incubated with factor Xa (1.6 nM, final) for 60 min.

Factor Xa Peptide Binding Assays: Peptide HC796-835 at 10 μg/mL in sodium bicarbonate (pH 9.3) was coated onto the wells of microtiter plates and then blocked with 5% fatty acid free BSA in TBS. After washing the plate with TBSA containing 5 mM CaCl₂, various concentrations of factor Xa in binding buffer consisting of TBSA containing 5 mM CaCl₂ were incubated in plate wells for 1 h at room temperature. Following 3 washings, bound factor Xa was detected by adding chromogenic substrate for factor Xa (S2222, Chromogenix, Franklin, Ohio). The absorbance values observed for triplicate noncoated wells lacking peptides served as nonspecific controls for binding and were subtracted from observed values for corresponding duplicate peptide-coated wells. Nonspecific binding of Factor Xa ranged from 5 to 20% of total observed binding in various experiments.

Thrombin generation assay in pooled human plasma: The effect of synthetic peptides on thrombin generation in pooled normal human plasma (George King Bio-medical, Inc., Overland Park, KS) was tested as described (1,11). Briefly, normal human plasma (30 μl) was mixed with CTI, followed by the addition of peptide. Then, tissue factor (TF) (Innovin) (0.5% pM final) containing 30 mM Ca⁺⁺ in TBSA with fluorogenic thrombin substrate solution (I-1140) (Bachem Americas, Inc., Torrance, Calif.) were added to the plasma to initiate coagulation activation. The first derivative of the time course data for substrate hydrolysis yielded the thrombin generation curve, allowing measurement of thrombin generation during the initiation, propagation and termination phases of thrombin generation and the value for peak thrombin generated.

Statistical analysis: Apparent Kd values using saturation binding curve fit were performed using Prism™ 7.0 software (Graph Pad Software Inc., San Diego, Calif.).

### Example 6

### Variant Peptides and Derivative Compounds Derived from HC 796-835 Peptide (SEQ ID NO:21)

A number of variants or derivatives of HC 796-835 peptide (SEQ ID NO:21) or peptide containing residues 816-835 (SEQ ID NO:24) were generated by rational design and various modifications. These include internal amino acid residue deletions, terminal truncations or additions of amino acid residues, conservative or non-conservative substitutions, substitutions with non-natural amino acid residue, and attachment of chemical compounds or moieties (“staples”). These variant peptides were tested for improved biological activities and/or pharmaceutical properties, as described herein. Activities of these peptides in comparison to the original HC 796-835 peptide (SEQ ID NO:21) are analyzed. Structures of the different chemical moieties (staples) in some of the variants are shown in FIG. 16. These variant peptides contain various substitutions with non-natural amino acid residues or D-amino acid residues. Structures of other substituting amino acid analogs are shown in FIG. 15.

Several assays were performed to assess anti-coagulation activities of these compounds, including assays using purified protein system and assays using recalcification of human plasma. Assays using purified protein system involve monitoring activation of prothrombin by prothrombinase complex containing factors Xa and Va and Ca++ ions. In one format (Assay 1A), skeletal muscle myosin was incubated with factor Va (5 nmol/L, final) and factor Xa (0.2 nmol/L, final) in TBS containing 0.5% BSA with 5 mmol/L Ca²⁺ in the presence of a test or candidate peptide in DMSO or DMSO alone. Thrombin generation was initiated by the addition of prothrombin (0.75 μM, final, unless noted otherwise). The reaction was quenched by 10 mmol/L EDTA at 10 min, and the rate of thrombin formation was quantified by measuring thrombin concentration as the rate of substrate (Pefachrome TH) hydrolysis or fluorogenic thrombin substrate solution (Z-Gly-Gly-Arg-AMC) (I-1140). For this assay 96 well plate (80 μL of myosin/Xa/Va and prothrombin mixture) or 384 well plates (30 μL of myosin/Xa/Va and prothrombin mixture) were used. IC₅₀s were determined using curve fit program in Prism7.0 (GraphPad Software, San Diego, Calif.).

In a second assay format (Assay 1B) of the purified protein system, no myosin was used. Instead, test peptide in DMSO or DMSO alone was incubated with the same reaction mixture of factor Xa, factor Va and prothrombin and Ca⁺⁺ in 96 well plate. The reaction was quenched by 10 mmol/L EDTA at 60 min, and the rate of thrombin formation was quantified by measuring thrombin concentration as the rate of substrate (Pefachrome TH) hydrolysis. IC₅₀ were determined using curve fit program in Prism7.0 (GraphPad Software, San Diego, Calif.). A third assay format (Assay 1C) of the purified protein system includes phospholipid. In this assay, candidate peptide in DMSO or DMSO alone was incubated with the same reaction mixture of factor Xa, factor Va and prothrombin, Ca⁺⁺ with phospholipid vesicle (4μM, final, phosphatidylserine 20%/phosphatidylcholine 80%) in 96 well plate. The reaction was quenched by 10 mmol/L EDTA at 20, 40 and 60 s in 96 well plate. The rate of thrombin formation was quantified by measuring thrombin concentration as the rate of substrate (Pefachrome TH) hydrolysis. IC₅₀s were determined using curve fit program in Prism7.0 (GraphPad Software, San Diego, Calif.).

Activities of the various peptides and derivative compounds were also examined via assays using recalcification of human plasma. [One of] these assay format (2A) involves monitoring thrombin generation in platelet poor plasma (PPP). Specifically, pooled normal human platelet poor plasma (PPP) (obtained from George King Bio Medical Inc., Overland Park, Kans.) (30%) was incubated with skeletal muscle myosin at 50 nM in the presence of peptide in DMSO or DMSO alone for 5 min at 37° C. Then, fluorogenic thrombin substrate solution (Z-Gly-Gly-Arg-AMC) (I-1140) with CaCl₂ (10 mmol/L final) alone was added to the reaction mixture (total 110 μl) to initiate coagulation. Thrombin generation was followed continuously for 25 min using SPECTRAmax GEMINI XS fluorometer (Molecular Devices, Sunnyvale, Calif.) with excitation and emission wavelengths set at 360 and 460 nm, respectively. The first derivative of fluorescence versus time was used to produce thrombin generation curves with the correction for substrate consumption and inner filter effect. For this assay 96 well plate (total 110 μL) or 384 well plates (total 30 μL) were used.

A variation of this assay format (Assay 2B) involves the same thrombin generation assay except no added myosin is present. Another assay format (Assay 3B) involves using recalcification of human plasma plus procoagulant tissue factor (TF). Specifically, to initiate coagulation in plasma, tissue factor (TF) (Innovin, final 0.5 pM) as well as CaCl₂ (10 mmol/L (final) were added to the plasma reaction mixture (total 100 μl) in the presence of skeletal muscle myosin (50 nM final). A further modification of this assay format (Assay 3A) involves the addition of myosin besides TF and Ca²⁺.

In addition to purified protein system and human plasma, assays using whole blood (Assay 4) were also performed to examine anti-coagulant activities of the peptides and derivative compounds. In these assays, freshly drawn anticoagulated human whole blood (80 μL aliquot) was incubated with peptide in DMSO or DMSO alone (control) for 5 min at 37° C. Then, fluorogenic thrombin substrate solution (Z-Gly-Gly-Arg-AMC) (I-1140) with 10 mmol/L CaCl₂ (final) alone was added to the reaction mixture (total 110 μl) in 96 well plate to initiate coagulation. Thrombin generation was followed continuously for 25 min using SPECTRAmax GEMINI XS fluorometer (Molecular Devices, Sunnyvale, Calif.) with excitation and emission wavelengths set at 360 and 460 nm, respectively. The first derivative of fluorescence versus time was used to produce thrombin generation curves with the correction for substrate consumption and inner filter effect. IC₅₀ were determined using a curve fit program in Prism7.0 (GraphPad Software, San Diego, Calif.).

Results obtained with the various assays noted above are indicated in Table 3. Activities obtained from Assay 2A (recalcification of human plasma in the presence of myosin) were used to rank the various peptides listed in Table 3. As noted above, this assay monitors recalcification of plasma to stimulate thrombin generation. In this assay, potency of the compounds was determined as the peptide concentration for 50% inhibition of thrombin generation, defined as “IC50”.

TABLE 3
Activities of variant peptides and derivative compounds
(as measured by different assays described in Example 6)

| | | | | 2A | | | 3B (+ | |
|---|---|---|---|---|---|---|---|---|
| | | 1B | | (recalcifica- | 2B | 3A TF/ | TF/Ca++ | 4 |
| | 1A (+my- | (no | | tion +my- | (recalcification | Ca++ +my- | no | (recalci- |
| | osin | myosin) | 1C (+phos- | osin) | no myosin) | osin) | myosin) | fication) |
| Cpd ID | (μM) | (μM) | pholipid | (μM) | (μM) | (μM) | (μM) | (μM) |
| HC426 | 0.17 | — | | 0.8 | | — | | |
| HC423 | 0.16 | — | | 0.96 | | — | | |
| HC421 | 0.12 | — | | 0.99 | | — | | |
| HC376 | 0.13 | — | | 1.1 | | — | | |
| SL- | 0.087 | — | | 1.15 | | — | | 0.7-5 |
| 1504- | | | | | | | | |
| 053A) | | | | | | | | |
| HC424 | 0.17 | — | | 1.2 | | — | | |
| HC364 | 0.063 | — | | 1.3 | | — | | 0.8-4 |
| SL- | 0.099 | — | | 1.3 | | — | | 0.3-3 |
| 1504- | | | | | | | | |
| 065A | | | | | | | | |
| SL- | 0.22 | — | | 1.3 | | | | Anticoagulant |
| 1504- | | | | | | | | |
| 086A | | | | | | | | |
| HC409 | 0.1 | — | | 1.4 | | — | | |
| HC425 | 0.17 | — | | 1.4 | | — | | |
| SL- | 0.1 | — | | 1.5 | | — | | Anticoagulant |
| 1504- | | | | | | | | |
| 064A | | | | | | | | |
| HC350 | 0.42 | — | | 1.5 | | — | | |
| HC413 | 0.094 | — | | 1.6 | | — | | |
| HC349 | 0.53 | — | | 1.6 | | — | | 4.9 |
| SL- | 0.044 | — | | 1.7 | | — | | 0.3-4 |
| 1504- | | | | | | | | |
| 074A | | | | | | | | |
| HC363) | 0.086 | — | | 1.7 | | — | | 0.4-4 |
| SL- | 0.087 | — | | 1.7 | | — | | 0.9-2 |
| 1504- | | | | | | | | |
| 062A | | | | | | | | |
| HC414 | 0.092 | — | | 1.7 | | — | | |
| HC419 | 0.097 | — | | 1.7 | | — | | |
| SL- | 0.1 | — | | 1.7 | | — | | Anticoagulant |
| 1504- | | | | | | | | |
| 063A) | | | | | | | | |
| HC374) | 0.19 | — | | 1.7 | | — | | |
| HC375 | 0.19 | — | | 1.7 | | — | | |
| HC422 | 0.12 | — | | 1.8 | | — | | |
| HC379 | 0.13 | — | | 1.8 | | — | | |
| HC357 | 0.18 | — | | 1.8 | | — | | |
| SL- | 0.17 | — | | 1.87 | | — | | 1.5-5 |
| 1504- | | | | | | | | |
| 051A | | | | | | | | |
| HC385 | 0.067 | — | | 1.9 | | — | | 0.5-5 |
| HC411 | 0.12 | — | | 1.9 | | — | | |
| HC373 | 0.18 | — | | 1.9 | | — | | |
| HC427 | 0.12 | — | | 2 | | — | | |
| HC420 | 0.16 | — | | 2.1 | | — | | |
| HC353 | 0.085 | — | | 2.2 | | — | | |
| HC408 | 0.13 | — | | 2.2 | | — | | |
| HC400 | 0.15 | — | | 2.2 | | — | | |
| HC355 | 0.18 | — | | 2.3 | | — | | |
| SL- | 0.25 | — | | 2.3 | | — | | Anticoagulant |
| 1504- | | | | | | | | |
| 069A | | | | | | | | |
| HC383 | 0.15 | — | | 2.4 | | — | | |
| HC356 | 0.16 | — | | 2.4 | | — | | |
| HC174 | 0.19 | — | | 2.4 | | — | | |
| SL- | 0.28 | — | | 2.4 | | — | | |
| 1504- | | | | | | | | |
| 090A | | | | | | | | |
| HC384 | 0.14 | — | | 2.5 | | — | | |
| HC381 | 0.22 | — | | 2.5 | | — | | |
| HC292 | 0.69 | — | | 2.5 | | — | | 0.5-4 |
| HC354 | 0.18 | — | | 2.6 | | — | | |
| SL- | 0.23 | — | | 2.6 | | — | | |
| 1504- | | | | | | | | |
| 082A | | | | | | | | |
| SL- | 0.55 | — | | 2.6 | | — | | 0.6-3.6 |
| 1504- | | | | | | | | |
| 068A | | | | | | | | |

TABLE 3
Activities of variant peptides and derivative compounds (continued)

| | | | | 2A | 2B (recalci- | 3A (+TF/ | 3B (+ | 4 |
|---|---|---|---|---|---|---|---|---|
| | 1A (+ | 1B (no | | (recalcifica | fication | Ca++ +my- | TF/Ca++ | (recalci- |
| | myosin | myosin) | 1C (+phos- | -tion +myosin) | no myosin) | osin) | no myosin) | fication) |
| Cpd ID | (μM)) | (μM) | pholipid | (μM) | (μM) | (μM) | (μM) | (μM) |
| HC394 | 0.12 | — | | 2.7 | | — | | |
| SL-1504- | 0.18 | — | | 2.7 | | — | | |
| 076A | | | | | | | | |
| HC372 | 0.17 | — | | 2.8 | | — | | |
| SL-1504- | 0.23 | — | | 2.8 | | — | | 4.5 |
| 071A | | | | | | | | |
| SL-1504- | 0.09 | — | | 2.9 | | — | | |
| 089A | | | | | | | | |
| HC365 | 0.16 | — | | 2.9 | | — | | |
| HC359 | 0.19 | — | | 2.9 | | — | | |
| HC393 | 0.23 | — | | 2.9 | | — | | |
| SL-1504- | 0.29 | — | | 2.9 | | — | | |
| 075A | | | | | | | | |
| SL-1504- | 0.51 | — | | 2.9 | | — | | 0.8-5 |
| 066A | | | | | | | | |
| HC412 | 0.093 | — | | 3.1 | | — | | |
| HC360 | 0.23 | — | | 3.1 | | — | | |
| HC392 | 0.35 | — | | 3.1 | | — | | |
| HC410 | 0.18 | — | | 3.2 | | — | | |
| HC362 | 0.14 | — | | 3.3 | | — | | |
| HC407 | 0.23 | — | | 3.3 | | — | | |
| HC282 | 0.51 | — | | 3.3 | | — | | |
| SL-1504- | 0.088 | — | | 3.4 | | — | | |
| 061A | | | | | | | | |
| HC361 | 0.14 | — | | 3.4 | | — | | |
| HC370 | 0.19 | — | | 3.4 | | — | | |
| HC358 | 0.24 | — | | 3.4 | | — | | |
| HC170 | 0.27 | — | | 3.4 | | — | | No activity |
| SL-1504- | 0.35 | — | | 3.4 | | — | | |
| 047A | | | | | | | | |
| HC160 | 0.75 | — | | 3.4 | | — | | |
| SL-1504- | 0.084 | — | | 3.5 | | — | | No activity |
| 049A | | | | | | | | |
| SL-1504- | 0.24 | — | | 3.5 | | — | | |
| 088A | | | | | | | | |
| SL-1504- | 0.32 | — | | 3.6 | | — | | |
| 067A | | | | | | | | |
| HC236 | 0.38 | — | | 3.6 | | — | | |
| HC395 | 0.12 | — | | 3.8 | | — | | |
| HC378 | 0.22 | — | | 3.8 | | — | | |
| HC391 | 0.12 | — | | 3.9 | | — | | |
| SL-1504- | 0.2 | — | | 4.3 | | — | | |
| 072A | | | | | | | | |
| SL-1504- | 0.58 | — | | 4.3 | | — | | |
| 070A | | | | | | | | |
| HC293 | 0.63 | — | | 4.4 | | — | | |
| HC084 | 0.45 | Myosin | | 4.5 | | Activity > | | |
| | | specific | | | | HC060 | | |
| HC380 | 0.15 | — | | 4.6 | | — | | |
| HC389 | 0.23 | — | | 4.8 | | — | | |
| HC417 | 0.14 | — | | 4.9 | | — | | |
| HC213 | 0.31 | — | | 4.9 | | — | | |
| HC216 | 0.92 | — | | 4.9 | | — | | |
| HC234 | 0.49 | — | | 5 | | — | | |
| HC399 | 0.2 | — | | 5.2 | | — | | |
| HC214 | 0.23 | — | | 5.3 | | — | | |
| SL-1504- | 0.36 | — | | 5.4 | | — | | |
| 077A | | | | | | | | |
| SL-1504- | 0.51 | — | | 5.4 | | — | | |
| 021A | | | | | | | | |
| HC285 | 0.54 | — | | 5.6 | | — | | |

TABLE 3
Activities of variant peptides and derivative compounds (continued)

| | | | | 2A | 2B (recalci- | | 3B (+TF/ | |
|---|---|---|---|---|---|---|---|---|
| | 1A (+ | 1B no | | (recalcifica- | fication | 3A (+TF/ | Ca++ | 4 (recalci- |
| | myosin | myosin) | 1C (+phos- | tion +my- | no myosin) | Ca++ +my- | no myosin) | fication) |
| Cpd ID | (μM)) | (μM) | pholipid | osin) (μM) | (μM) | osin) (μM) | (μM) | (μM) |
| HC158 | 0.44 | — | | 5.8 | | — | | |
| HC199 | 0.46 | — | | 5.8-10.2 | | — | | |
| SL- | 2.7 | — | | 5.9 | | — | | |
| 1504- | | | | | | | | |
| 048A | | | | | | | | |
| HC087 | 0.64 | Myosin | | 6.0 | | Activity | | |
| | | specific | | | | > HC060 | | |
| SL- | 1.5 | — | | 6 | | — | | |
| 1504- | | | | | | | | |
| 052A | | | | | | | | |
| HC166 | 0.54 | — | | 6.4 | | — | | |
| HC161 | 0.69 | — | | 6.4 | | — | | |
| HC283 | 0.49 | — | | 6.6 | | — | | |
| HC310 | 0.15 | — | | 6.7 | | — | | |
| HC225 | 0.52 | — | | 6.8 | | — | | |
| HC284 | 0.49 | — | | 7 | | — | | |
| HC223 | 0.88 | — | | 7.1 | | — | | |
| HC152 | 0.9 | — | | 7.4 | | — | | |
| HC226 | 0.76 | — | | 7.7 | | — | | |
| HC325 | 0.26 | — | | 7.8 | | — | | |
| HC313 | 0.55 | — | | 7.8 | | — | | |
| SL- | 1 | — | | 8 | | — | | |
| 1504- | | | | | | | | |
| 016A | | | | | | | | |
| (HC202) | | | | | | | | |
| HC097 | 1.7 | Myosin | | 8 | | — | | |
| | | specific | | | | | | |
| HC179 | 0.4 | — | | 8.3 | | — | | No activity |
| SL- | 0.56 | — | | 8.6 | | — | | |
| 1504- | | | | | | | | |
| 017A | | | | | | | | |
| (HC202) | | | | | | | | |
| HC281 | 0.24 | — | | 8.7 | | — | | |
| HC265 | 0.3 | — | | 8.7 | | — | | |
| HC224 | 1.4 | — | | 8.7 | | — | | |
| SL- | 0.72 | — | | 8.9 | | — | | |
| 1504- | | | | | | | | |
| 011A | | | | | | | | |
| (HC201) | | | | | | | | |
| HC101 | 0.32- | Myosin | | 9 | | Activity > | | |
| | 1.3 | specific | | | | HC061 | | |
| SL- | 0.63 | — | | 9.1 | | — | | |
| 1504- | | | | | | | | |
| 022A | | | | | | | | |
| (HC232) | | | | | | | | |
| HC098 | 0.60 | Myosin | | 9.3 | | Activity > | | |
| | | specific | | | | HC061 | | |
| HC156 | 0.64 | — | | 9.3 | | — | | |
| HC154 | 1.1 | — | | 9.4 | | — | | |
| HC155 | 0.83 | — | | 9.5 | | — | | |
| HC139 | 4.7 | — | | 9.7 | | — | | |
| HC188 | 0.59 | — | | 10 | | — | | |
| HC167 | 0.83 | — | | 10 | | — | | |
| SL- | 0.89 | — | | 10 | | — | | |
| 1504- | | | | | | | | |
| 015B | | | | | | | | |
| (HC202) | | | | | | | | |
| HC095 | 1.1 | Myosin | | 10 | | Activity = | | |
| | | specific | | | | HC061 | | |
| SL- | 4 | 10 | | 10 | | — | | |
| 1504- | | | | | | | | |
| 029A | | | | | | | | |
| (HC201) | | | | | | | | |
| HC173 | 0.97 | — | | 10.5 | | — | | |
| HC153 | 0.39 | — | | 11 | | — | | |
| HC157 | 0.65 | — | | 11 | | — | | |
| SL- | 0.3 | — | | 12.7 | | — | | |
| 1504- | | | | | | | | |
| 041A | | | | | | | | |
| HC178 | 1.7 | — | | 13 | | — | | |
| HC159 | 0.74 | — | | 13.3 | | — | | |
| HC077 | 0.91 | Myosin | | 15 | | Activity = | | |
| | | specific | | | | HC061 | | |
| HC176 | 0.95 | — | | 15 | | — | | |
| HC198 | 0.87 | — | | 16 | | — | | |
| HC189 | 1.5 | — | | 17 | | — | | |
| HC080 | 0.8 | Myosin | | 20 | | Activity = | | |
| | | specific | | | | HC061 | | |

TABLE 3
Activities of variant peptides and derivative compounds (continued)

| | | | | 2A | 2B (recalci- | | 3B (+TF/ | |
|---|---|---|---|---|---|---|---|---|
| | 1A (+ | 1B no | | (recalcifica- | fication | 3A (+TF/ | Ca++ | 4 (recalci- |
| | myosin | myosin) | 1C (+phos- | tion +my- | no myosin) | Ca++ +my- | no myosin) | fication) |
| Cpd ID | (μM)) | (μM) | pholipid | osin) (μM) | (μM) | osin) (μM) | (μM) | (μM) |
| HC060 | 1.1 | Myosin | No activity | 20 | | Activity = | | |
| | | specific | | | | HC061 | | |
| HC063 | Activity << | Myosin | | < HC060 | | < HC060 | | |
| | HC061 | specific | | | | | | |
| HC064 | Activity < | Myosin | | = HC060 | | Activity > | | |
| | HC061 | specific | | | | HC060 | | |
| HC096 | 2.7 | Myosin | | Activity < | | — | | |
| | | specific | | HC095 | | | | |
| HC134 | 0.9 | Myosin | | Activity < | | Activity < | | |
| | | specific | | HC061 | | HC061 | | |
| HC136 | 1.6 | — | | Activity < | | Activity < | | |
| | | | | HC061 | | HC061 | | |
| HC133 | 1.8 | Myosin | | Activity < | | Activity < | | |
| | | specific | | HC061 | | HC061 | | |
| HC128 | 2 | Myosin | | Activity < | | Activity < | | |
| | | specific | | HC061 | | HC061 | | |
| HC130 | 2.1 | Myosin | | Activity < | | Activity < | | |
| | | specific | | HC061 | | HC061 | | |
| HC131 | 2.2 | Myosin | | Activity < | | Activity < | | |
| | | specific | | HC061 | | HC061 | | |
| HC135 | 2.7 | — | | Activity < | | Activity < | | |
| | | | | HC061 | | HC061 | | |
| HC132 | 3.1 | Myosin | | Activity < | | Activity < | | |
| | | specific | | HC061 | | HC061 | | |
| HC129 | 4.2 | Myosin | | Activity < | | Activity < | | |
| | | specific | | HC061 | | HC061 | | |
| HC126 | 4.9 | Myosin | | Activity < | | Activity < | | |
| | | specific | | HC061 | | HC061 | | |
| HC137 | 5.5 | — | | Activity < | | Activity < | | |
| | | | | HC061 | | HC061 | | |
| HC127 | 5.9 | Myosin | | Activity < | | Activity < | | |
| | | specific | | HC061 | | HC061 | | |
| HC100 | Activity < | Myosin | | Activity < | | Activity < | | |
| | HC061 | specific | | HC061 | | HC061 | | |
| HC056 | Activity << | Myosin | | Activity < | | Activity < | | |
| | HC061 | specific | | HC061 | | HC061 | | |
| HC058 | Activity << | Myosin | | Activity < | | Activity < | | |
| | | specific | | HC061 | | HC061 | | |
| SL- | Activity > | Myosin | | Activity << | | Activity < | | |
| 1504- | HC061 | specific | | HC061 | | HC061 | | |
| 001 | | | | | | | | |
| HC123 | 0.6 | Myosin | | Activity << | | Activity << | | |
| | | specific | | HC061 | | HC061 | | |
| HC125 | 0.6 | Myosin | | Activity << | | Activity << | | |
| | | specific | | HC061 | | HC061 | | |
| HC124 | 0.7 | Myosin | | Activity << | | Activity << | | |
| | | specific | | HC061 | | HC061 | | |
| HC115 | 0.72 | Myosin | | Activity << | | Activity << | | |
| | | specific | | HC061 | | HC061 | | |
| HC117 | 0.78 | Myosin | | Activity << | | Activity << | | |
| | | specific | | HC061 | | HC061 | | |
| HC116 | 0.85 | Myosin | | Activity << | | Activity << | | |
| | | specific | | HC061 | | HC061 | | |
| HC114 | 0.98 | Myosin | | Activity << | | Activity << | | |
| | | specific | | HC061 | | HC061 | | |
| HC122 | 1.1 | Myosin | | Activity << | | Activity << | | |
| | | specific | | HC061 | | HC061 | | |
| HC118 | 1.8 | Myosin | | Activity << | | Activity << | | |
| | | specific | | HC061 | | HC061 | | |
| HC121 | 3.2 | Myosin | | Activity << | | Activity << | | |
| | | specific | | HC061 | | HC061 | | |
| HC119 | 5.2 | Myosin | | Activity << | | Activity << | | |
| | | specific | | HC061 | | HC061 | | |
| HC120 | 5.2 | Myosin | | Activity << | | Activity << | | |
| | | specific | | HC061 | | HC061 | | |
| HC099 | Activity = | Myosin | | Activity << | | Activity << | | |
| | HC061 | specific | | HC061 | | HC061 | | |
| HC104 | Activity = | Myosin | | Activity << | | Activity << | | |
| | HC061 | specific | | HC061 | | HC061 | | |
| HC094 | 0.4 | Myosin | | Activity = | | Activity = | | |
| | | specific | | HC061 | | HC061 | | |
| HC093 | 0.7 | Myosin | | Activity = | | Activity = | | |
| | | specific | | HC061 | | HC061 | | |
| HC004 | 1.3 | Not | anticoagulant | Activity = | Activity > | anticoagulant | anticoagulant | |
| | | specific | | HC061 | HC061 | | | |
| HC092) | 1.5 | Myosin | | Activity = | | Activity = | | |
| | | specific | | HC061 | | HC061 | | |
| HC078 | Activity < | Myosin | | Activity = | | Activity = | | |
| | HC061 | specific | | HC061 | | HC061 | | |
| HC065 | Activity = | Myosin | No activity | Activity = | | Activity = | | |
| | HC061 | specific | | HC061 | | HC061 | | |
| HC088 | Activity = | Myosin | | Activity = | | Activity = | | |
| | HC061 | specific | | HC061 | | HC061 | | |
| HC090 | Activity > | Myosin | | Activity = | | Activity = | | |
| | HC061 | specific | | HC061 | | HC061 | | |
| HC079 | Activity < | Myosin | | Activity > | | Activity > | | |
| | HC061 | HC061 | | HC061 | | HC061 | | |
| HC085 | Activity > | Myosin | | Activity > | | Activity > | | |
| | HC061 | HC061 | | HC061 | | HC061 | | |
| HC102 | Activity > | Myosin | | Activity > | | Activity > | | |
| | HC061 | HC061 | | HC061 | | HC061 | | |
| HC103 | Activity > | Myosin | | Activity > | | Activity > | | |
| | HC061 | HC061 | | HC061 | | HC061 | | |
| HC327 | 0.24 | — | | — | | — | | |

TABLE 3
Activities of variant peptides and derivative compounds (continued)

| | | | | 2A | 2B | | | |
|---|---|---|---|---|---|---|---|---|
| | 1A (+ | 1B no | | (recalcifica- | fication | 3A (+TF/ | Ca++ | 4 (recalci- |
| | myosin | myosin) | 1C (+phos- | tion +my- | no myosin) | Ca++ +my- | no myosin) | fication) |
| Cpd ID | (μM)) | (μM) | pholipid | osin) (μM) | (μM) | osin) (μM) | (μM) | (μM) |
| HC268 | 0.31 | — | | — | | — | | |
| HC351 | 0.32 | — | | — | | — | | |
| HC309 | 0.41 | — | | — | | — | | |
| HC294 | 0.42 | — | | — | | — | | |
| HC336 | 0.43 | — | | — | | — | | |
| HC286 | 0.49 | — | | — | | — | | |
| HC296 | 0.49 | — | | — | | — | | |
| HC333 | 0.49 | — | | — | | — | | |
| HC246 | 0.53 | — | | — | | — | | |
| SL-1504-033B | 0.54 | — | | — | | — | | |
| SL-1504- | 0.58 | — | | — | | — | | |
| 038A | | | | | | | | |
| HC326 | 0.58 | — | | — | | — | | |
| HC335 | 0.61 | — | | — | | — | | |
| HC334 | 0.63 | — | | — | | — | | |
| HC270 | 0.65 | — | | — | | — | | |
| HC304 | 0.69 | — | | — | | — | | |
| HC211 | 0.7 | — | | — | | — | | |
| SL-1504- | 0.7 | — | | — | | — | | |
| 018A | | | | | | | | |
| (HC229) | | | | | | | | |
| HC298 | 0.71 | — | | — | | — | | |
| HC244 | 0.78 | — | | — | | — | | |
| SL-1504- | 0.78 | — | | — | | — | | |
| 039A | | | | | | | | |
| HC297 | 0.79 | — | | — | | — | | |
| HC253 | 0.8 | — | | — | | — | | |
| HC263 | 0.8 | — | | — | | — | | |
| HC247 | 0.81 | — | | — | | — | | |
| HC295 | 0.81 | — | | — | | — | | |
| HC230 | 0.83 | — | | — | | — | | |
| HC209 | 0.9 | — | | — | | — | | |
| HC237 | 0.93 | — | | — | | — | | |
| SL-1504- | 0.94 | — | | — | | — | | |
| 034A | | | | | | | | |
| HC264 | 0.95 | — | | — | | — | | |
| HC229 | 0.96 | — | | — | | — | | |
| HC239 | 0.96 | — | | — | | — | | |
| HC299 | 0.99 | — | | — | | — | | |
| HC210 | 1 | — | | — | | — | | |
| HC254 | 1 | — | | — | | — | | |
| HC328 | 1 | — | | — | | — | | |
| HC185 | 1.1 | — | | — | | — | | |
| HC255 | 1.1 | — | | — | | — | | |
| HC332 | 1.1 | — | | — | | — | | |
| HC194 | 1.2 | — | | — | | — | | |
| HC207 | 1.2 | — | | — | | — | | |
| HC222 | 1.2 | — | | — | | — | | |
| HC266 | 1.2 | — | | — | | — | | |
| SL-1504- | 1.3 | — | | — | | — | | |
| 019A | | | | | | | | |
| (HC256) | | | | | | | | |
| SL-1504- | 1.3 | — | | — | | — | | |
| 020A (212) | | | | | | | | |
| (HC257) | | | | | | | | |
| HC252 (258) | 1.3 | — | | — | | — | | |

TABLE 3
Activities of variant peptides and derivative compounds (continued)

| | | | | 2A | 2B | | | |
|---|---|---|---|---|---|---|---|---|
| | 1A (+ | 1B no | | (recalcifica- | fication | 3A (+TF/ | Ca++ | 4 (recalci- |
| | myosin | myosin) | 1C (+phos- | tion +my- | no myosin) | Ca++ +my- | no myosin) | fication) |
| Cpd ID | (μM)) | (μM) | pholipid | osin) (μM) | (μM) | osin) (μM) | (μM) | (μM) |
| SL- | 1.3 | — | | — | | — | | |
| 1504- | | | | | | | | |
| 037A | | | | | | | | |
| HC200 | 1.4 | — | | — | | — | | |
| HC208 | 1.4 | — | | — | | — | | |
| SL- | 1.4 | — | | — | | — | | |
| 1504- | | | | | | | | |
| 013A | | | | | | | | |
| (HC201) | | | | | | | | |
| HC259 | 1.4 | — | | — | | — | | |
| HC272 | 1.4 | — | | — | | — | | |
| HC248 | 1.5 | — | | — | | — | | |
| HC251 | 1.5 | — | | — | | — | | |
| HC267 | 1.5 | — | | — | | — | | |
| HC269 | 1.5 | — | | — | | — | | |
| HC279 | 1.5 | — | | — | | — | | |
| SL- | 1.5 | — | | — | | — | | |
| 1504- | | | | | | | | |
| 032B | | | | | | | | |
| HC191 | 1.6 | — | | — | | — | | |
| HC215 | 1.6 | — | | — | | — | | |
| SL- | 1.6 | — | | — | | — | | |
| 1504- | | | | | | | | |
| 010A | | | | | | | | |
| (HC201) | | | | | | | | |
| SL- | 1.6 | — | | — | | — | | |
| 1504- | | | | | | | | |
| 031B | | | | | | | | |
| HC195 | 1.7 | — | | — | | — | | |
| HC262 | 1.7 | — | | — | | — | | |
| HC271 | 1.7 | — | | — | | — | | |
| HC206 | 1.8 | — | | — | | — | | |
| HC212 | 1.8 | — | | — | | — | | |
| HC348 | 1.8 | — | | — | | — | | |
| HC204 | 1.9 | — | | — | | — | | |
| HC217 | 1.9 | — | | — | | — | | |
| HC221 | 1.9 | — | | — | | — | | |
| HC276 | 1.9 | — | | — | | — | | |
| HC312 | 1.9 | — | | — | | — | | |
| SL- | 2 | — | | — | | — | | |
| 1504- | | | | | | | | |
| 012A | | | | | | | | |
| (HC201) | | | | | | | | |
| HC329 | 2.1 | — | | — | | — | | |
| HC192 | 2.2 | — | | — | | — | | |
| HC280 | 2.2 | — | | — | | — | | |
| HC219 | 2.3 | — | | — | | — | | |
| HC249 | 2.3 | — | | — | | — | | |
| HC250 | 2.3 | — | | — | | — | | |
| HC301 | 2.3 | — | | — | | — | | |
| HC172 | 2.4 | — | | — | | — | | |
| HC258 | 2.4 | — | | — | | — | | |
| HC311 | 2.4 | — | | — | | — | | |
| HC257 | 2.5 | — | | — | | — | | |
| SL- | 2.6 | — | | — | | — | | |
| 1504- | | | | | | | | |
| 036A | | | | | | | | |
| HC300 | 2.7 | — | | — | | — | | |
| HC321 | 2.7 | — | | — | | — | | |
| HC203 | 2.8 | — | | — | | — | | |
| HC273 | 2.9 | — | | — | | — | | |
| HC274 | 3 | — | | — | | — | | |
| HC322 | 3 | — | | — | | — | | |
| SL- | 3.1 | — | | — | | — | | |
| 1504- | | | | | | | | |
| 050A | | | | | | | | |

TABLE 3
Activities of variant peptides and derivative compounds (continued)

| | | | | 2A | 2B | | | |
|---|---|---|---|---|---|---|---|---|
| | 1A (+ | 1B no | | (recalcifica- | fication | 3A (+TF/ | Ca++ | 4 (recalci- |
| | myosin | myosin) | 1C (+phos- | tion +my- | no myosin) | Ca++ +my- | no myosin) | fication) |
| Cpd ID | (μM)) | (μM) | pholipid | osin) (μM) | (μM) | osin) (μM) | (μM) | (μM) |
| HC205 | 3.2 | — | — | — | | — | | |
| HC227 | 3.5 | — | — | — | | — | | |
| HC190 | 3.6 | — | — | — | | — | | |
| HC228 | 3.6 | — | — | — | | — | | |
| HC261 | 3.6 | — | — | — | | — | | |
| HC277 | 3.6 | — | — | — | | — | | |
| SL- | 3.9 | — | — | — | | — | | |
| 1504- | | | | | | | | |
| 046A | | | | | | | | |
| HC260 | 4.1 | — | — | — | | — | | |
| HC275 | 4.2 | — | — | — | | — | | |
| HC291 | 4.7 | — | — | — | | — | | |
| HC245 | 4.8 | — | — | — | | — | | |
| HC150 | 5 | — | — | — | | — | | |
| HC256 | 8.4 | — | — | — | | — | | |
| HC278 | 8.5 | — | — | — | | — | | |
| HC317 | 0.54 | — | — | — | | — | | |
| HC220 | 0.83 | — | | Partial | | — | | |
| | | | | inhibition | | | | |
| HC001 | 0.96 | Myosin | No activity | No activity | Procoagulant | No activity | | |
| | | specific | | | | | | |
| HC012 | 10 | >15 | No activity | No activity | No activity | No activity | | |
| HC008 | 15 | 20 | No activity | No activity | >20 | No activity | | |
| HC014 | 15 | 15 | No activity | No activity | Activity < | No activity | | |
| | | | | | HC061 | | | |
| HC162 | >10 | — | | No activity | | No activity | | |
| HC163 | >10 | — | | No activity | | No activity | | |
| HC382 | 0.17 | — | | | | | | |
| HC315 | 0.27 | — | | | | | | |
| HC415 | 0.28 | — | | | | | | |
| HC366 | 0.29 | — | | | | | | |
| HC369 | 0.3 | — | | | | | | |
| HC371 | 0.3 | — | | | | | | |
| HC418 | 0.3 | — | | | | | | |
| HC377 | 0.31 | — | | | | | | |
| HC367 | 0.32 | — | | | | | | |
| SL- | 0.34 | — | | | | | | |
| 1504- | | | | | | | | |
| 073A | | | | | | | | |
| HC416 | 0.4 | — | | | | | | |
| HC368 | 0.41 | — | | | | | | |
| HC396 | 0.43 | — | | | | | | |
| SL- | 0.46 | — | | | | | | |
| 1504- | | | | | | | | |
| 092A | | | | | | | | |
| HC398 | 0.53 | — | | | | | | |
| HC390 | 0.65 | — | | | | | | |
| HC397 | 0.68 | — | | | | | | |
| SL- | 0.79 | — | | | | | | |
| 1504- | | | | | | | | |
| 084A | | | | | | | | |
| HC386 | 0.93 | — | | | | | | |
| HC387 | 0.98 | — | | | | | | |
| HC402 | 1.1 | — | | | | | | |
| HC401 | 1.3 | — | | | | | | |
| HC429 | 1.3 | — | | | | | | |
| SL- | 1.4 | — | | | | | | |
| 1504- | | | | | | | | |
| 091A | | | | | | | | |
| HC404 | 2.1 | — | | | | | | |

TABLE 3
Activities of variant peptides and derivative compounds (continued)

| | | | | 2A | 2B | | 3B (+ TF/ | 4 |
|---|---|---|---|---|---|---|---|---|
| | 1A | 1B | | (recalcifica- | (recalcification | 3A (+TF/ | Ca++ no | (recalci- |
| | myosin | (no myosin) | 1C (+phos- | tion +my- | no myosin) | Ca++ +my- | myosin) | fication) |
| Cpd ID | (μM)) | (μM) | pholipid | osin) (μM) | (μM) | osin) (μM) | (μM) | (μM) |
| HC388 | 10 | — | | | | | | |
| HC059 | | Myosin | No activity | | | | | |
| | | specific | | | | | | |
| HC290 | | | | | | | | |
| HC403 | | | | | | | | |
| HC405 | | | | | | | | |
| HC406 | | | | | | | | |
| HC061 | 1.3 | Activity < | No activity | 3.8 (partial) | | Activity < | | No activity |
| | | Peptide | | | | Peptide | | |
| | | 20 | | | | 20 | | |
| HC815- | 1.99 | 10.8 | 7.5 | | | | | |
| 854 | | | | | | | | |
| RLC133- | 25 | | Myosin | | | No | | |
| 162 | | | specific | | | activity | | |
| ELC138- | 75 | | | | | | | |
| 157 | | | | | | | | |
| ELC | ~70 | | | | | | | |
| 129-144 | | | | | | | | |
| ELC109- | 70 | | Myosin | | | No | | |
| 138 | | | specific | | | activity | | |
| ELC129- | 100 | | Myosin | | | No | | |
| 159 | | | specific | | | activity | | |
| 7a | — | — | | | | | | |
| 7h | — | — | | | | | | |
| 7c | — | — | | | | | | |
| 7d | — | — | | | | | | |
| 7e | — | — | | | | | | |
| ELC 12a | — | — | | | | | | |
| ELC 13a | — | — | | | | | | |
| ELC 16 | — | — | | | | | | |
| ELC 16a | — | — | | | | | | |

### Example 7

### Overall Strategy for Optimizing Myosin Derived Anti-Coagulant Peptides

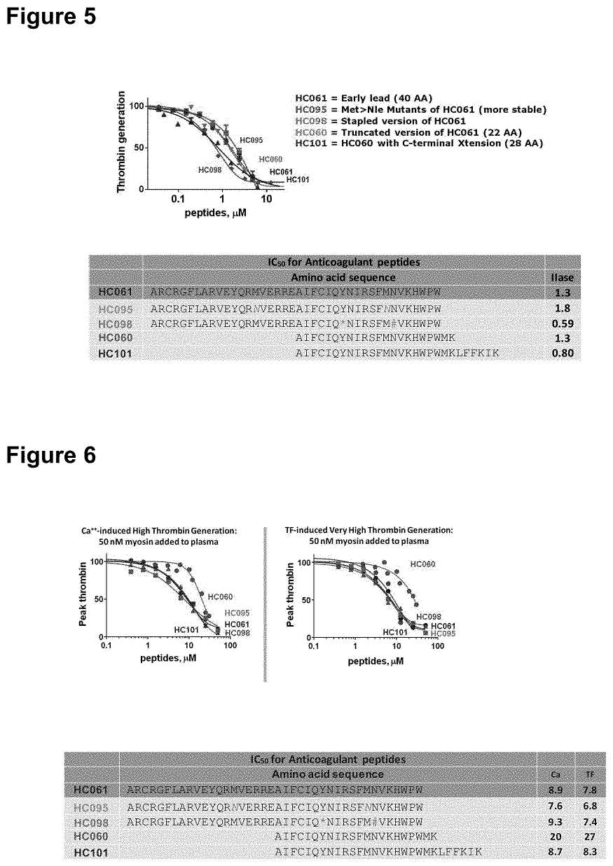

Peptide HC061 (SEQ ID NO:21) has an IC50 of 1.3 micromolar in the in vitro assay, demonstrating prothrombin activation by factor Xa/factor Va/myosin in purified system (FIG. 5). Peptide HC095 was synthesized as a more stable analog of HC061 by replacement of two methionine residues with norleucines since norleucine cannot be oxidized as methionine residues can be. Peptide HC098 is a stapled version of HC061. Peptide stapling is used to increase peptide stability by stabilizing an alpha helical secondary conformation. HC060 was identified by truncations of HC061 to identify the smallest peptide with equivalent activity. HC101 was designed by C-terminal extension of HC060 to enhance activity.

A secondary in vitro screen was also used to rank the lead peptides. The secondary assay utilized addition of Ca⁺⁺-induced high Thrombin generation and TF-induced very high thrombin generation in plasma spiked with 50 nM myosin. The secondary assay results for the initial lead compounds are shown in FIG. 6.

Alanine scanning of HC060 demonstrated the critical residues needed for anticoagulant activity. Mutation of the underlined residues causes a loss of activity of HC060: AIFCIQYNIRSFMNVKHWPWMK-NH2 (SEQ ID NO:97). FIG. 7 shows the alanine scanning results.

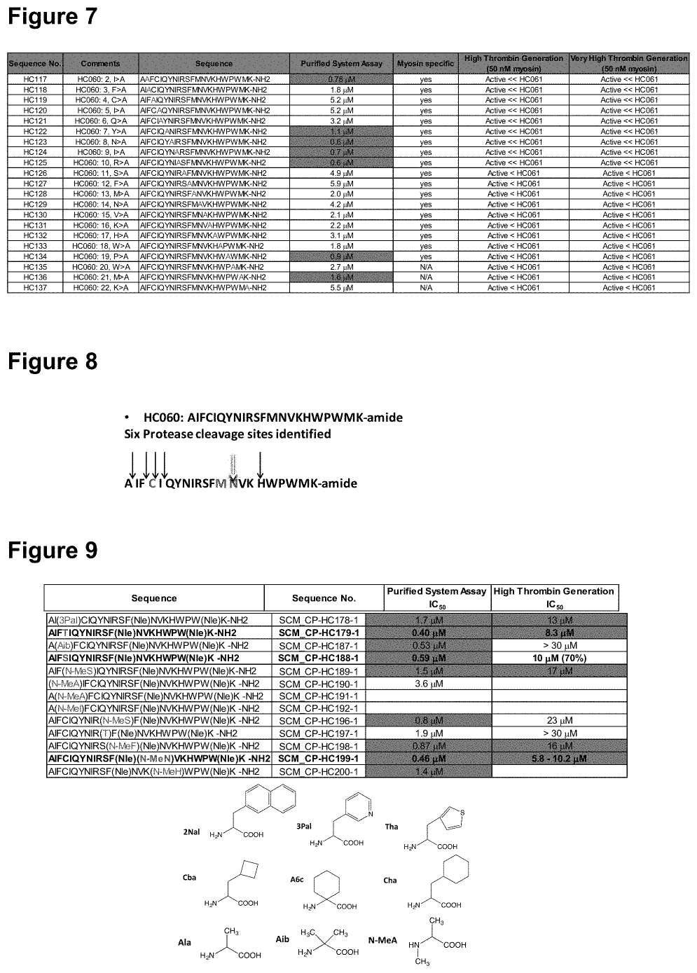

A series of unnatural amino acids were substituted in place of those residues identified by alanine scanning to be important to activity (FIG. 9). The objective was to find unnatural amino acid replacements that would both improve activity and peptide stability. FIG. 9 shows the results from select unnatural amino acid replacements. The substitution of cysteine (C) with threonine (T) at position4 improved activity, and substitution of asparagine (N) with N-Methyl asparagine (N-MeN) at position 14 improved activity and stability. The design/placement of N-Methyl asparagines at position 14 was also based on the plasma stability data, which showed this as a site susceptible to protease cleavage as illustrated in FIG. 8.

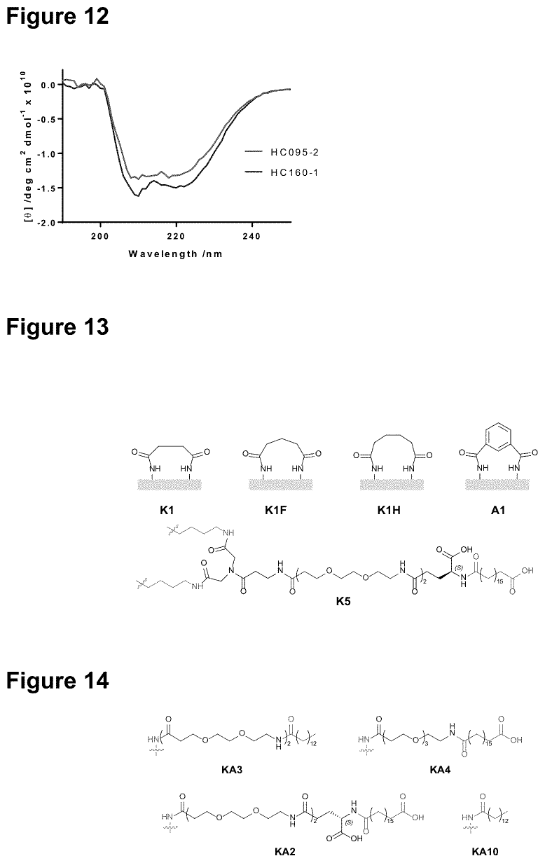

Attachment of lipid / fatty acid moieties to the candidate sequences was utilized in order to improve their half-lives via enhancing binding to circulating serum albumin. As lead peptides were likely to be alpha-helical (based on myosin complex crystal structure) peptide stapling was applied simultaneously in order to enhance peptide helicity and enhance lead potency (FIG. 12). This approach utilized dual stapling and lipid conjugation methodology that were described in, e.g., PCT publications WO2015/095406 and WO2016/205488. Optimization of staple length was carried out using ‘simple’ staples K1, K1F, K1H and A1, before incorporation of the lipid moiety (e.g. K5) (FIG. 13). Stapling (‘K’ and ‘A’ series) was not found to offer significant activity enhancement in this case however, and single-site lipid conjugation was instead pursued (‘KA’ series). Lipid structure—including PEG linker length, fatty acid chain length and decoration with carboxylate groups—and placement within the sequence was adjusted to optimize conjugate potency (FIG. 14).

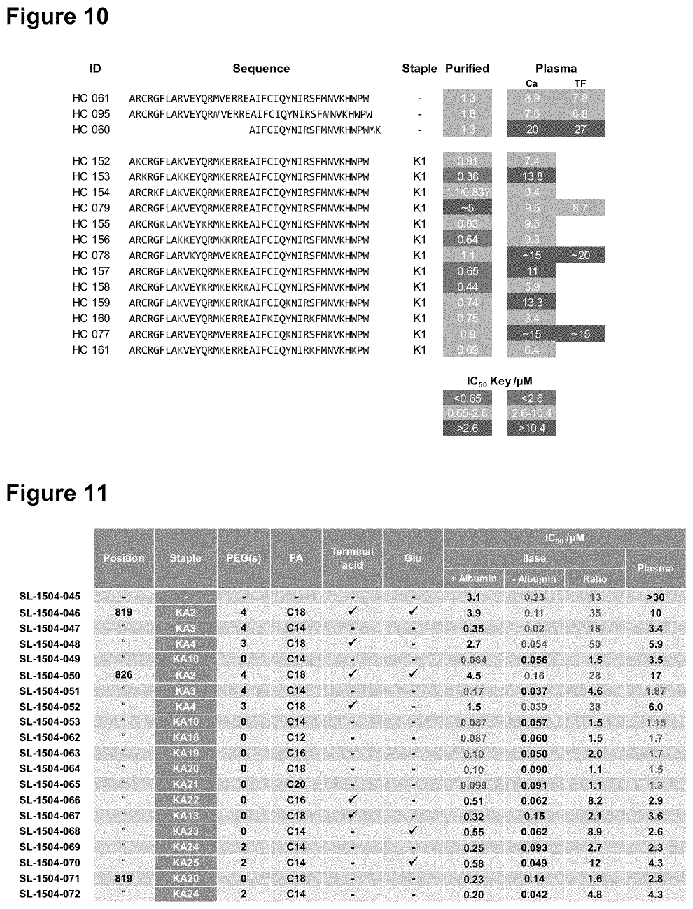

Activities of peptide derived from HC061 sequence stapled with K1 staple at various positions are shown in FIG. 10. Effect of lipid structure and position on conjugate activity is shown in FIG. 11. A panel of conjugates bearing structurally diverse lipids was also screened in a whole blood thrombin generation assay to assess the effect on anticoagulant activity. Effect of sequence, lipid structure and position on conjugate activity in whole blood was analyzed.

In summary, the key peptide optimization strategies utilized include the following: Incorporation of unnatural amino acids based on Ala scan results to improve peptide potency; N- and C-terminal truncations of lead peptides: looking for the optimal sequence length; Design of hybrid peptides combining features from the most active peptides; Methionine and Cysteine (residues prone to oxidation) replacements for increased stability; Aib and N-Methyl amino acid scan at the amino terminus and other critical positions for increased activity and stability; D-amino acid scan; N-terminal capping with various acids; C-terminal Lys5 tail to test the hypothesis that hydrophilicity may reduce plasma protein binding and increase the amount of free peptide; New stapled peptide analogs based on promising staple positions for increased peptide stability.

### Example 8

### Synthesis of Chemical Moieties (Staples) for Modifying Peptides

Unless otherwise noted, all reagents were purchased from commercial suppliers (Sigma Aldrich, Fisher, Oakwood) and used without further purification. All reactions involving air or moisture sensitive reagents or intermediates were performed under an inert atmosphere of nitrogen or argon. All solvents used were of HPLC grade.

Peptides were synthesized by standard solid-phase peptide synthesis (SPPS) techniques and purified via HPLC (as described below).

Flash chromatography purifications were performed on silica gel prepacked columns (40 μm, RediSep® Rf from Teledyne Isco) on a CombiFlash® Rf (Teledyne Isco). Purified final compounds were eluted as single and symmetrical peaks (thereby confirming a purity of ≥95%).

Semi-preparative chromatography were performed on a Shimadzu HPLC with a Phenomenex Luna column (C18, 100 Å pore size, 10 μm particle size, 250×10.0 mm, flow: 4 mL/min) or on an Agilent 1200 HPLC with a Phenomenex Luna column (C18, 100 Å pore size, 5 μm particle size, 150×21.2 mm, flow: 20 mL/min).

¹H and ¹³C NMR spectra were recorded on a Bruker 400 system in d₆-DMSO, CDCl₃ or CD₃OD. Chemical shifts are given in parts per million (ppm) with tetramethylsilane as an internal standard. Abbreviations are used as follows: s=singlet, d=doublet, t=triplet, q=quartet, p=pentet, m=multiplet, dd=doublet of doublets, br=broad. Coupling constants (values) are given in hertz (Hz).

Low resolution mass spectra were recorded on a Waters Acquity UPLC with a Phemomenex Luna Omega C18 column (C18, 100 Å pore size, 1.6 μm particle size, 50×2.1 mm, flow: 0.4 mL/min). Solvents: A—H₂O+0.1% formic acid, B—MeCN+0.1% formic acid, gradient: 0-1 min 10-90% B, 1-1.6 min 90% B, 1.6-1.7 min 90-10% B, 1.7-2 min 10% B.

High resolution mass spectra (HRMS) were recorded on an Agilent 1200 Series Accurate Mass Time-of-Flight (TOF) with an Aeris Widepore column (XB-C8, 3.6 μm particle size, 150×2.1 mm, flow: 0.5 mL/min). Solvents: A—H₂O+0.1% formic acid, B—MeCN+0.1% formic acid, gradient: 0-2 min 5% B, 2-12 min 5-60% B, 12-13 min 60-80% B, 13-14 min 80-20% B, 14-15 min 20-80% B, 15-16 min 80-20% B, 16-17 min 20-95% B, 17-20 min 95% B, 20-21 min 95-5% B.

### Example 9

### Methods for Attaching Chemical Moieties to Peptides (Stapling)

General solid-phase protocols for lactam stapling: Peptide-resin bearing amine side chain orthogonal protection (Dde/Mmt) at each stapling position was swollen in DMF for 1 h. The Dde protecting group was removed from the first side chain via treatment with 2% hydrazine solution in DMF (2×15 min). Positive TNBS test was observed. The linker building block specified below was coupled as described and a negative TNBS test was observed. The solvent was exchanged for DCM and the Mmt group was removed from the second side chain via treatment with 1% TFA in DCM containing 5% TIPS, 5×2 min. The resin was washed with DCM, 10% DIEA in DMF, DMF and a positive TNBS test was observed. The linker was cyclized and the PEG-fatty acid portion of the staple (if applicable) elongated as described below. The complete stapled peptide was cleaved from the resin using 95% TFA, 2.5% TIPS, 2.5% H₂O, 3 h. The peptide cleavage mixture was evaporated to an oil, triturated and washed with diethyl ether and purified via reversed-phase HPLC.

A Dde/Alloc protection scheme can also be used for this approach, which requires the addition of allyl alcohol to the Dde deprotection cocktail as a scavenger to prevent concurrent reduction of the Alloc allyl moiety.

General protocol for ‘A1’ and ‘K1’ series simple lactam staples: For linker coupling the appropriate diacid building block (2 eq) was attached using HATU (4 eq) and DIEA (4 eq) in DMF, 1×2 h. The cyclization step was achieved using HATU (1 eq) and DIEA (2 eq) in DMF, 1×2 h.

General protocol for ‘K’ PEG-fatty acid trifunctional lactam staples: For linker coupling the intramolecular symmetric anhydride of building block K(Fmoc) linker (2 eq) was preformed using DIC (2 eq) and catalytic DMAP in dry DCM for 10 min at RT. The peptide-resin solvent was exchanged for DCM and the anhydride was then added and agitated overnight. The resin was drained, washed with DCM and DMF. The linker was cyclized overnight via treatment with DIC (1 eq) and HOBt or HOAt (1 eq) in DMF, and a negative TNBS was observed. Remaining uncyclized linker was capped via treatment with 10% acetic anhydride in DMF (30 min). The linker Fmoc group was deprotected via treatment with 20% piperidine in DMF (2×10 min). A positive TNBS was observed. Subsequent staple PEG and fatty acid building blocks were attached sequentially to the linker free amine via standard coupling chemistry: building block (3 eq), HATU (3 eq) and DIEA (6 eq) in DMF, 1 h at RT, using 20% piperidine in DMF for deprotection cycles (5 +10 min, RT).

General protocol for ‘A’ PEG-fatty acid trifunctional lactam staples: For linker coupling the building block A(Fmoc) linker (2 eq) was attached using HATU (4 eq) and DIEA (4 eq) in DMF, 1×2 h. The cyclization step was achieved using HATU (1 eq) and DIEA (2 eq) in DMF, 1×2 h. Remaining uncyclized linker was capped via treatment with 10% acetic anhydride in DMF (30 min). The linker Fmoc group was deprotected via treatment with 20% piperidine in DMF (2×10 min). It was not possible to observe a positive TNBS test for the aniline nitrogen. Fmoc-β-Ala-OH (3 eq) was coupled using HATU (3 eq) and DIEA (6 eq) in DMF, 4×1 h at RT or as the symmetric anhydride using DIC/DMAP in DCM (2 h, RT). Subsequent staple PEG and fatty acid building blocks were attached sequentially to the linker free amine via standard coupling chemistry: building block (3 eq), HATU (3 eq) and DIEA (6 eq) in DMF, 1 h at RT, using 20% piperidine in DMF for deprotection cycles (5+10 min, RT).

Synthesis of K(Fmoc) linker:

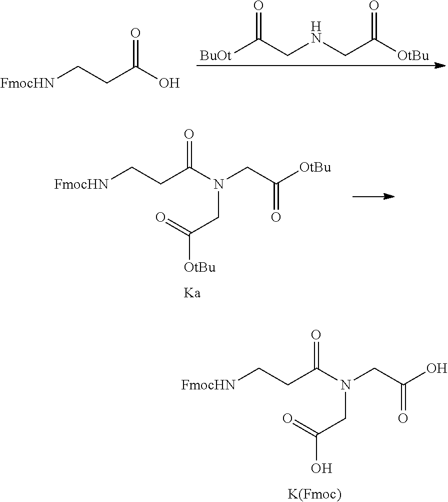

Intermediate Ka: Fmoc-β-Ala-OH (1.00 g, 3.21 mmol) and di-tent-butyl iminodiacetate (0.461 g, 2.68 mmol) were suspended in 100 mL DCM. HATU (1.02 g, 2.68 mmol) and DIEA (3.32 mL, 12.8 mmol) were added and the reaction was stirred at RT for 3.5 h. The solvent was evaporated and the residue dissolved in MeOH and purified via flash column chromatography on silica gel (hexane/EtOAc) to afford the product as a white solid (0.802 g, 56%).

¹H NMR (400 MHz, chloroform-d) δ 7.78 (d, J=7.4 Hz, 2H), 7.62 (d, J=7.4 Hz, 2H), 7.42 (t, J=7.4 Hz, 2H), 7.33 (t, J=7.4 Hz, 2H), 5.66 (t, J=5.7 Hz, 1H), 4.35 (d, J=7.3 Hz, 2H), 4.23 (t, J=7.3 Hz, 1H), 4.10 (s, 2H), 4.02 (s, 2H), 3.56 (q, J=5.7 Hz, 2H), 2.55 (t, J=5.7 Hz, 2H), 1.49 (s, 18H).

K(Fmoc) linker: Compound Ka was treated with 20 mL 1:1 TFA/DCM for 2 h. The solvent was evaporated and the residue triturated and washed with diethyl ether to afford K(Fmoc) linker as a white solid (0.371 g, 58%).j

MS (ES⁺) m/z 427.15 ([M+H]⁺)

Synthesis of A(Fmoc) linker:

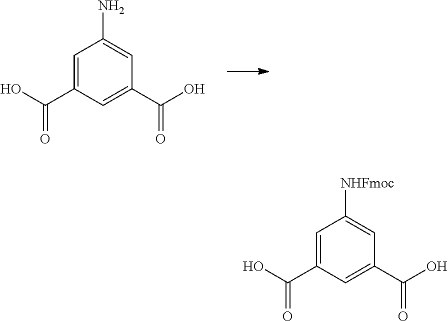

A solution of 5-Aminoisophthalic acid (1.00 g, 5.5 mmol) in 10 mL dioxane was added to a degassed solution of Na₂CO₃ (1.46 g, 5.5 mmol) in 15 mL water. The solution was cooled on ice and a solution of Fmoc chloride (1.42 g, 5.5 mmol) in 10 mL dioxane was then added dropwise with stirring over 15 min. The reaction was then stirred for 1 h and then 24 h at RT. The dioxane was removed under vacuum and the remaining aqueous solution acidified with 1M HCl. The resulting solid precipitate was then washed with diethyl ether (4×10 mL), redissolved in EtOAc, filtered, washed with brine, dried over Na₂SO₄ filtered and concentrated to give A(Fmoc) linker as a white solid.

¹H NMR (500 MHz, DMSO-d₆) δ 13.24 (s, 2H), 10.12 (s, 1H), 8.33 (d, J=1.5 Hz, 2H), 8.12 (t, J=1.5 Hz, 1H), 7.91 (d, J=7.6 Hz, 2H), 7.76 (dd, J=7.6, 1.2 Hz, 2H), 7.43 (t, J=7.6 Hz, 2H), 7.36 (td, J=7.6, 1.2 Hz, 2H), 4.50 (d, J=6.8 Hz, 2H), 4.33 (t, J=6.8 Hz, 1H).

Additional notes on stapling (chemical modifying) the various peptides. Any staple beginning ‘KA’ was attached at one point (with one ‘X’ in the sequence), all other staples were attached at two points (with two ‘X’s to form a cycle). The staple structure (given in the Word document) was attached to the side chain nitrogen of the ‘X’ residue(s) for each modified compound (e.g. Lys, Orn or N-terminus). In some peptides, there is simply a disulfide formed between two Cys residues, with no other chemical moiety (staple) attached. Similarly, some other peptides have one or two residues replaced with amino acid analogs R8 and/or S8, which are able to form a double bond (all hydrocarbon “stapling”) via Grubbs/ring-closing metathesis (RCM) reaction without any further chemical moiety (staple) added. See, e.g., Walensky et al., J. Med. Chem. 57:6275-6288, 2014. Structures of the various staples or chemical moieties used to modify the compounds are shown in FIG. 16. Unless otherwise noted, a “KA” staple refers to single-site lipidation. The ‘A1’ and ‘K1’ cyclic staples have two attachment points, unlike ‘KA’ staples. Trifunctional' staples refer to the remaining two-site staples (i.e. not ‘K1’, ‘A1’, ‘KA’ series) as they attach at two points on the peptide, but also have a third ‘branch’ for the lipid to attach to (see e.g. ‘K5’ staples). In addition, lactam stapling' refers to any of attachments carried out using amide bond-forming chemistry (i.e. any ‘K’, ‘A’, ‘KA’ series), but not any disulfide or ‘all hydrocarbon’ (‘R’ or ‘S’)

Other than K1 staples, other “K” staple refers to two-site stapling to form a cycle using the ‘K(Fmoc)’ linker. Similarly, except ‘A1’ staples, other “A” staple is the same as a “K” staple except that an aromatic ‘A(Fmoc)’ linker is used. Additionally, any ‘K1’ or ‘A1’ cyclic staples (e.g. ‘K1H’, ‘A1’ etc., but not ‘K10’, ‘K12’, ‘A10’ etc.) do not have any fatty acid appendage attached (“simple cyclic staples”), whereas the others do have fatty acid appendage attached to the indicated staple (chemical moiety) itself. Thus, ‘A1’ and ‘K1’ series ‘simple’ staples do not use the ‘K(Fmoc)’ or ‘A(Fmoc)’ linker for synthesis since a third attachment point isn't needed. Instead they use only the simple diacid (without Fmoc group)—e.g. compare ‘A1’ and ‘A10’, or ‘K1F’ and ‘K5’.

### Example 10

### Synthesis and Purification of an Exemplary Derivative Compound

This example describes synthesis and purification of one of the derivative myosin peptide that was chemically modified. This compound contains a chemical moiety, “KA21”, that was attached to a Lys residue in the derivative peptide sequence shown in SEQ ID NO:98 (AIFSIQYNIRXF(Nle)(N-MeN)VKHWPW(Nle)KKKKK).

Preparation of peptide SL-1504-074A (SEQ ID NO:98):

Commercially available MBHA resin (0.21 mmol/g, 2.4 g) was charged in the reaction vessel, and the peptide: Fmoc-Lys(Boc)-OH (468.5 mg, 1 mmol), Fmoc-Lys(Boc)-OH (468.5 mg, 1 mmol), Fmoc-Lys(Boc)-OH (468.5 mg, 1 mmol), Fmoc-Lys(Boc)-OH (468.5 mg, 1 mmol), Fmoc-Nle-OH (353.4 mg, 1 mmol), Fmoc-Trp(Boc)-OH (526.6 mg, 1 mmol), Fmoc-Pro-OH (337 mg, 1 mmol), Fmoc-Trp(Boc)-OH (526. 6 mg, 1 mmol), Fmoc-His(Trt)-OH (619.7 mg, 1 mmol), Fmoc-Trp(Boc)-OH (526.6 mg, 1 mmol), Fmoc-Val-OH (339.4 mg, 1 mmol), Fmoc-NMe-Asn(Trt)-OH (610.7 mg, 1 mmol), Fmoc-Nle-OH (353.4 mg, 1 mmol), Fmoc-Phe-OH (387 mg, 1 mmol), Fmoc-Lys(Dde)-OH (532.6 mg, 1 mmol), Fmoc-Arg(Pdf)-OH (648.8 mg, 1 mmol), Fmoc-Ile-OH (353.4 mg, 1 mmol), Fmoc-Asn(Trt)-OH (596.7 mg, 1 mmol), Fmoc-Tyr(tBu)-OH (459 mg, 1 mmol), Fmoc-Gln(Trt)-OH (610 mg, 1 mmol), Fmoc-Ile-OH (353.4 mg, 1 mmol), Fmoc-Ser(tBu)-OH (383 mg, 1 mmol), Fmoc-Phe-OH (387 mg, 1 mmol), Fmoc-Ile-OH (353.4 mg, 1 mmol), Boc-Ala-OH(189.2 mg, 1 mml) were sequentially introduced in the order given above by the Fmoc-strategy (HBTU-HOBt with DIEA in DMF, for 0.1 M) peptide synthesis process to give the objective protected peptide resin, except the second Fmoc-Nle-OH was coupled by DIC-HOBt for 2 days. All the deprotection was made with piperidine 20% in DMF. After peptide chain enlongation, the Dde protecting group was removed by adding 4% Hydrazine hydrate/DMF (15 mL, 15 min) for 3 times. And then, Arachidic Acid (312.5 mg, 1 mmol) in DMF solution was added with HBTU-HOBt-DIEA. The reaction mixture was incubated with resin for 1 hr, then washed out and did again.

After solid phase assembly, cleavage from the resin and concomitant side chain deprotection with a mixture of TFA/Thioanisole/Tis/H2O (90:4:3:3, v/v/v/v) for 4 hr at room temperature. The resin was filtered and then washed with TFA twice. The cold MTBE (675 mL) was added drop-wise to the combined TFA cleavage solution and the precipitate was taken by filtration. The precipitate was then washed with cold MTBE four times, and dry under vacuum. 1.2 of crude peptide was obtained.

Purification is performed by a preparative reverse phase HPLC:

1. The 1^{st} purification by using TFA buffer (A: 0.1% TFA in water; B: 0.1% TFA in acetonitril). Column: XBridge Peptide BEH C18, 19*250 mm, 10 μm; Eluants: elution on a lineal density gradient of A/B=55/45−43/57 (25 mins). A 1.2 g aliquot (in 35 mL of acetonitril-water mixture) was purified on HPLC to give 300 mg of the target peptide with purity 95%.

2. The 2^{nd} purification by using TFA buffer (A: 0.1% TFA in water; B: 0.1% TFA in acetonitril). Column: XBridge Peptide BEH C18, 19*150 mm, 5 μm; Eluants: elution on a lineal density gradient of A/B=80/20−20/80 (20 mins); Flow Rate: 15 mL/min; A 300 mg aliquot (in 75 mL of acetonitril-water mixture) was purified on HPLC to give 110 mg of the target peptide with purity 99%.

3. The 3^{rd} purification by using Acetic acid buffer (A: 1% HOAc in water; B: 1% HOAc in acetonitril). Column:)(Bridge Peptide BEH C18, 19*150 mm, 5 μm; Eluants: elution on a lineal density gradient of AB=95/5−35/65 (20 mins); Flow Rate: 15 mL/min; A 110 mg aliquot in 25 ml of acetonitril-water mixture, was adjusted pH to 9.3 by adding NH4*H2O solution before loading to the column. After purification, 70 mg of target peptide (HOAc salt) with 99% purity was obtained.

### Example 11

### Inhibition of Cardiac Myosin-Enhanced Prothrombin Activation

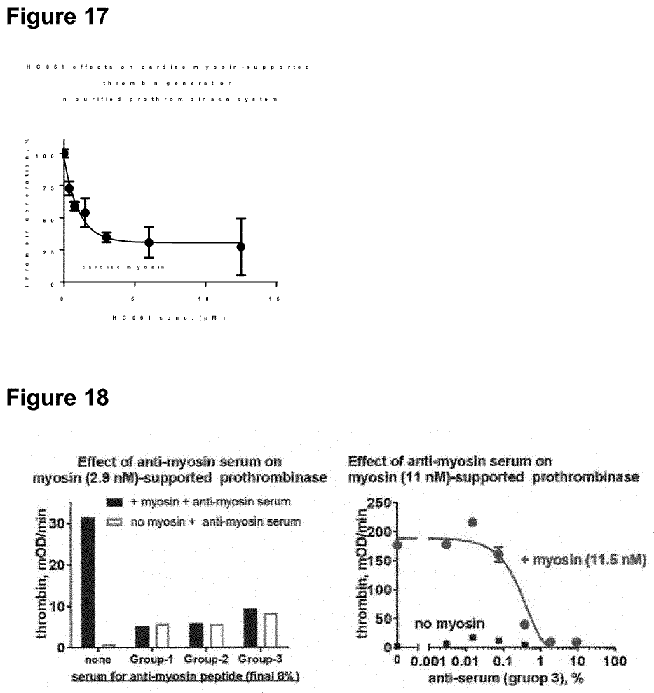

FIG. 17 shows data for inhibition of cardiac myosin's procoagulant activity. Varying concentrations of peptide HC796-835 (SEQ ID NO:21), ranging from 0 to 12.5 μM, were incubated with factor Va (5 nM, final) and factor Xa (0.2 nM, final) in Tris buffered saline containing bovine serum albumin (TBSA) plus 5 mM CaCl₂ at room temperature in the presence of cardiac myosin (2 nM, final). Thrombin generation was initiated by the addition of prothrombin (0.75 μM) in TB SA containing 5 mM CaCl₂. The reaction was quenched by adding EDTA (10 mM final) at 10 min. The rate of thrombin formation was quantified by measuring thrombin concentration as the rate of thrombin substrate hydrolysis. 100% was the value for controls in the absence of added peptides. Each value represents the mean [±SEM] of triplicate determinations.

### Example 12

### Inhibition of Prothrombin Activation by Antibodies Against Peptide HC060

This Example describes the use of murine antisera from mice immunized with the myosin derived peptides described herein to inhibition myosin-dependent prothrombin activation. The results are shown in FIG. 18. Specifically, the peptides of SEQ ID NO:97 (HC060) with an additional Cys residues at either the C-terminus (Group 1) or the N-terminus were synthesized, and Keyhole Lympet Hemocyanin (KLH) was conjugated at the end cysteine for immunogen of anti-myosin antibodies. The two peptide immunogens were injected into SJL mice for groups 1 and 2, respectively, and both peptides were injected into SJL mice comprising group 3. Antisera dilutions (1:12) from each mice group were tested and shown to inhibit very significantly myosin's enhancement of prothrombin generation with purified clotting factor assay protocols. Dose-response studies for antisera from group 3 mice showed that this antiserum blocked myosin's enhancement of prothrombin activation when the antiserum was present at >2%, i.e., that it was not diluted more than 1:50. Thus, anti-myosin peptide antisera blocked the procoagulant activity of myosin.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. A myosin derived anti-coagulant peptide, comprising the sequence as shown in any one of SEQ ID NOs:10, 23 and 24, or a substantially identical or conservatively modified sequence thereof.

2. The peptide of claim 1, comprising the sequence as shown in any one of SEQ ID NOs:16, 21, 22 and 28-96, or a substantially identical or conservatively modified sequence thereof.

3. The peptide of claim 1, comprising one or more substitutions with non-natural amino acid residues, amino acid analogs or D-amino acid residues.

4. The peptide of claim 1, further comprising an engineered disulfide bond or a engineered hydrocarbon bond.

5. The peptide of claim 1, further comprising an N-terminal acylation.

6. The peptide of claim 1, further comprising a C-terminal poly-lysine tail that consists of about 4 to about 8 lysine residues.

7. The peptide of claim 1, further comprising a chemical moiety (staple) that is attached to one or two residues in the sequence of the peptide.

8. The peptide of claim 7, wherein the one or two residues are internal residues in the sequence of the peptide.

9. The peptide of claim 7, wherein the chemical moiety is attached to the N-terminus or the C-terminus of the peptide.

10. The peptide of claim 7, wherein the chemical moiety is further attached to a lipid moiety.

11. The peptide of claim 1, which possesses an inhibitory activity on myosin mediated prothrombin activation.

12. A pharmaceutical composition, comprising the myosin derived anti-coagulant peptide of claim 1 and a pharmaceutically acceptable carrier.

13. A polynucleotide sequence that encodes the peptide of claim 1.

14. A derivative compound of a myosin derived anti-coagulant peptide, comprising a modified variant peptide of SEQ ID NO:24 (HC816-835) or SEQ ID NO:21 (HC061).

15. The derivative compound of claim 14, wherein the variant peptide comprises deletion of one or more terminal or internal residues of SEQ ID NO:24.

16. The derivative compound of claim 14, wherein the variant peptide comprises one or more amino acid substitutions in SEQ ID NO:24 with non-natural amino acid residues, amino acid analogs or D-amino acid residues.

17. The derivative compound of claim 14, wherein the variant peptide comprises SEQ ID NO:24 with C-terminal additions of (1) an Nle residue and (2) a poly-lysine tail that consists of about 4 to about 8 lysine residues.

18. The derivative compound of claim 14, wherein the variant peptide comprises SEQ ID NO:24 that is modified by conjugating a chemical moiety (staple) to one or two residues in the sequence of the peptide.

19. The derivative compound of claim 14, wherein the variant peptide is modified by one or more of the following substitutions in SEQ ID NO:24: Cys⁴→Lys, Thr or Ser, Ser¹¹→Lys, Met¹³→Nle, and Asn¹⁴→N-MeN.

20. The derivative compound of claim 18, wherein the chemical moiety is attached to an engineered Lys residue that substitutes for Ser¹¹ in SEQ ID NO:24.

21. The derivative compound of claim 20, wherein the chemical moiety is KA21, KA10, KA26, KA18, KA19, KA3, KA24, KA23, KA27 or KA22 as shown in FIG. 16.

22. The derivative compound of claim 18, wherein a lipid or fatty acid moiety is conjugated to the chemical moiety.

23. The derivative compound of claim 14, which possesses an inhibitory activity on myosin mediated prothrombin activation.

24. A pharmaceutical composition, comprising the derivative compound of claim 14 and a pharmaceutically acceptable carrier.

25. A method for treating or preventing undesired thrombosis in a subject, comprising administering to the subject a pharmaceutical composition that comprises a therapeutically effect amount of (1) a myosin derived anti-coagulant peptide that comprises the sequence as shown in any one of SEQ ID NOs:10, 23 and 24, or a substantially identical or conservatively modified sequence thereof or (2) a derivative compound of a myosin derived anti-coagulant peptide that comprises a modified variant peptide of SEQ ID NO:24 (HC816-835) or SEQ ID NO:21 (HC061), thereby treating or preventing undesired thrombosis in the subject.

26. The method of claim 25, wherein the undesired thrombosis is supported or mediated by myosin.

27. The method of claim 25, wherein the subject is an acute trauma patient.

28. The method of claim 25, wherein the subject has or is at risk of developing acute trauma coagulopathy.

29. The method of claim 26, wherein the subject has or is at risk of developing coronary thrombosis.


SEQUENCE LISTING (98 sequences)

SEQ ID NO: 1 - protein, 16 aa, Artificial sequence
  Synthetic sequence
  MOD_RES (1)..(1): ACETYLATION
  MOD_RES (16)..(16): AMIDATION
YEDFVEGLRVFDKEGN

SEQ ID NO: 2 - protein, 20 aa, Artificial sequence
  Synthetic sequence
  MOD_RES (1)..(1): ACETYLATION
  MOD_RES (20)..(20): AMIDATION
RVFDKEGNGTVMGAELRHVL

SEQ ID NO: 3 - protein, 18 aa, Artificial sequence
  Synthetic sequence
  MOD_RES (1)..(1): ACETYLATION
  MOD_RES (18)..(18): AMIDATION
GTVMGAELRHVLATLGEK

SEQ ID NO: 4 - protein, 35 aa, Artificial sequence
  Synthetic sequence
  MOD_RES (1)..(1): ACETYLATION
  MOD_RES (35)..(35): AMIDATION
DQTQIQEFKEAFTVIDQNRDGIIDKEDLRDTFAAM

SEQ ID NO: 5 - protein, 34 aa, Artificial sequence
  Synthetic sequence
  MOD_RES (1)..(1): ACETYLATION
  MOD_RES (34)..(34): AMIDATION
VIDQNRDGIIDKEDLRDTFAAMGRLNVKNEELDA

SEQ ID NO: 6 - protein, 28 aa, Artificial sequence
  Synthetic sequence
  MOD_RES (1)..(1)
  MOD_RES (28)..(28): AMIDATION
KNEELDAMMKEASGPINFTVFLTMFGEK

SEQ ID NO: 7 - protein, 27 aa, Artificial sequence
  Synthetic sequence
  MOD_RES (1)..(1): ACETYLATION
  MOD_RES (27)..(27): AMIDATION
NFTVFLTMFGEKLKGADPEDVITGAFK

SEQ ID NO: 8 - protein, 31 aa, Artificial sequence
  Synthetic sequence
  MOD_RES (1)..(1): ACETYLATION
  MOD_RES (31)..(31): AMIDATION
PEDVITGAFKVLDPEGKGTIKKKFLEELLTT

SEQ ID NO: 9 - protein, 25 aa, Artificial sequence
  Synthetic sequence
  MOD_RES (1)..(1): ACETYLATION
  MOD_RES (25)..(25): AMIDATION
FLEELLTTQCDRFSQEEIKNMWAAF

SEQ ID NO: 10 - protein, 30 aa, Artificial sequence
  Synthetic sequence
  MOD_RES (1)..(1): ACETYLATION
  MOD_RES (30)..(30): AMIDATION
QEEIKNMWAAFPPDVGGNVDYKNICYVITH

SEQ ID NO: 11 - protein, 34 aa, Artificial sequence
  Synthetic sequence
  MOD_RES (1)..(1): ACETYLATION
  MOD_RES (34)..(34): AMIDATION
KEQQDEFKEAFLLFDRTGDSKITLSQVGDVLRAL

SEQ ID NO: 12 - protein, 22 aa, Artificial sequence
  Synthetic sequence
  MOD_RES (1)..(1): ACETYLATION
  MOD_RES (22)..(22): AMIDATION
QVGDVLRALGTNPTNAEVRKVL

SEQ ID NO: 13 - protein, 18 aa, Artificial sequence
  Synthetic sequence
  MOD_RES (1)..(1): ACETYLATION
  MOD_RES (18)..(18): AMIDATION
TNAEVRKVLGNPSNEELN

SEQ ID NO: 14 - protein, 24 aa, Artificial sequence
  Synthetic sequence
  MOD_RES (1)..(1): ACETYLATION
  MOD_RES (24)..(24): AMIDATION
SNEELNAKKIEFEQFLPMMQAISN

SEQ ID NO: 15 - protein, 30 aa, Artificial sequence
  Synthetic sequence
  MOD_RES (1)..(1): ACETYLATION
  MOD_RES (30)..(30): AMIDATION
EFEQFLPMMQAISNNKDQATYEDFVEGLRV

SEQ ID NO: 16 - protein, 31 aa, Artificial sequence
  Synthetic sequence
  MOD_RES (1)..(1): ACETYLATION
  MOD_RES (31)..(31): AMIDATION
YEDFVEGLRVFDKEGNGTVMGAELRHVLATL

SEQ ID NO: 17 - protein, 25 aa, Artificial sequence
  Synthetic sequence
  MOD_RES (1)..(1): ACETYLATION
  MOD_RES (25)..(25): AMIDATION
GAELRHVLATLGEKMKEEEVEALMA

SEQ ID NO: 18 - protein, 60 aa, Artificial sequence
  Synthetic sequence
  MOD_RES (1)..(1): ACETYLATION
  MOD_RES (60)..(60): AMIDATION
KEEEVEALMAGQEDSNGCINYEAFVKHIMSKEEEVEALMAGQEDSNGCINYEAFVKHIMS

SEQ ID NO: 19 - protein, 38 aa, Artificial sequence
  Synthetic sequence
  MOD_RES (38)..(38): AMIDATION
LYADFKQRYKVLNASAIPEGQFIDSKKASEKLLASIDI

SEQ ID NO: 20 - protein, 30 aa, Artificial sequence
  Synthetic sequence
  MOD_RES (30)..(30): AMIDATION
EMRDDKLAQLITRTQARCRGFLARVEYQRM

SEQ ID NO: 21 - protein, 40 aa, Artificial sequence
  Synthetic sequence
  MOD_RES (40)..(40): AMIDATION
ARCRGFLARVEYQRMVERREAIFCIQYNIRSFMNVKHWPW

SEQ ID NO: 22 - protein, 40 aa, Artificial sequence
  Synthetic sequence
  MOD_RES (40)..(40): AMIDATION
EAIFCIQYNIRSFMNVKHWPWMKLFFKIKPLLKSAETEKE

SEQ ID NO: 23 - protein, 10 aa, Artificial sequence
  Synthetic sequence
YEDFVEGLRV

SEQ ID NO: 24 - protein, 20 aa, Artificial sequence
  Synthetic sequence
AIFCIQYNIRSFMNVKHWPW

SEQ ID NO: 25 - protein, 1941 aa, Homo sapiens
MSSDSELAVFGEAAPFLRKSERERIEAQNRPFDAKTSVFVAEPKESFVKGTIQSREGGKVTVKTEGGATLTVKDDQVFPMNPPKYDKIEDMAMMTHLHEPAVLYNLKERYAAWMIYTYSGLFCVTVNPYKWLPVYKPEVVTAYRGKKRQEAPPHIFSISDNAYQFMLTDRENQSILITGESGAGKTVNTKRVIQYFATIAVTGEKKKEEITSGKIQGTLEDQIISANPLLEAFGNAKTVRNDNSSRFGKFIRIHFGTTGKLASADIETYLLEKSRVVFQLKAERSYHIFYQITSNKKPELIEMLLITTNPYDYPFVSQGEISVASIDDQEELMATDSAIDILGFTNEEKVSIYKLTGAVMHYGNLKFKQKQREEQAEPDGTEVADKAAYLQSLNSADLLKALCYPRVKVGNEYVTKGQTVEQVSNAVGALAKAVYEKMFLWMVARINQQLDTKQPRQYFIGVLDIAGFEIFDFNSLEQLCINFTNEKLQQFFNHHMFVLEQEEYKKEGIEWTFIDFGMDLAACIELIEKPMGIFSILEEECMFPKATDTSFKNKLYDQHLGKSANFQKPKVVKGKAEAHFALIHYAGVVDYNITGWLEKNKDPLNETVVGLYQKSAMKTLAQLFSGAQTAEGEGAGGGAKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLASIDIDHTQYKFGHTKVFFKAGLLGLLEEMRDDKLAQLITRTQARCRGFLARVEYQRMVERREAIFCIQYNIRSFMNVKHWPWMKLFFKIKPLLKSAETEKEMATMKEEFQKIKDELAKSEAKRKELEEKMVTLLKEKNDLQLQVQAEAEGLADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKIKLEQQVDDLEGSLEQEKKLRMDLERAKRKLEGDLKLAQESIMDIENEKQQLDEKLKKKEFEISNLQSKIEDEQALGIQLQKKIKELQARIEELEEEIEAERASRAKAEKQRSDLSRELEEISERLEEAGGATSAQIEMNKKREAEFQKMRRDLEEATLQHEATAATLRKKHADSVAELGEQIDNLQRVKQKLEKEKSEMKMEIDDLASNVETVSKAKGNLEKMCRTLEDQLSELKSKEEEQQRLINDLTAQRGRLQTESGEFSRQLDEKEALVSQLSRGKQAFTQQIEELKRQLEEEIKAKNALAHALQSSRHDCDLLREQYEEEQESKAELQRALSKANTEVAQWRTKYETDAIQRTEELEEAKKKLAQRLQAAEEHVEAVNAKCASLEKTKQRLQNEVEDLMLDVERTNAACAALDKKQRNFDKILAEWKQKCEETHAELEASQKEARSLGTELFKIKNAYEESLDQLETLKRENKNLQQEISDLTEQIAEGGKRIHELEKIKKQVEQEKCELQAALEEAEASLEHEEGKILRIQLELNQVKSEVDRKIAEKDEEIDQLKRNHIRIVESMQSTLDAEIRSRNDAIRLKKKMEGDLNEMEIQLNHANRMAAEALRNYRNTQGILKDTQIHLDDALRSQEDLKEQLAMVERRANLLQAEIEELRATLEQTERSRKIAEQELLDASERVQLLHTQNTSLINTKKKLETDISQMQGEMEDILQEARNAEEKAKKAITDAAMMAEELKKEQDTSAHLERMKKNMEQTVKDLQLRLDEAEQLALKGGKKQIQKLEARVRELEGEVESEQKRNAEAVKGLRKHERRVKELTYQTEEDRKNILRLQDLVDKLQAKVKSYKRQAEEAEEQSNTNLAKFRKLQHELEEAEERADIAESQVNKLRVKSREVHTKVISEE

SEQ ID NO: 26 - protein, 194 aa, Homo sapiens
MAPKKDVKKPVAAAAAAPAPAPAPAPAPAPAKPKEEKIDLSAIKIEFSKEQQDEFKEAFLLFDRTGDSKITLSQVGDVLRALGTNPTNAEVRKVLGNPSNEELNAKKIEFEQFLPMMQAISNNKDQATYEDFVEGLRVFDKEGNGTVMGAELRHVLATLGEKMKEEEVEALMAGQEDSNGCINYEAFVKHIMSI

SEQ ID NO: 27 - protein, 169 aa, Homo sapiens
MAPKRAKRRTVEGGSSSVFSMFDQTQIQEFKEAFTVIDQNRDGIIDKEDLRDTFAAMGRLNVKNEELDAMMKEASGPINFTVFLTMFGEKLKGADPEDVITGAFKVLDPEGKGTIKKKFLEELLTTQCDRFSQEEIKNMWAAFPPDVGGNVDYKNICYVITHGDAKDQE

SEQ ID NO: 28 - protein, 23 aa, Artificial sequence
  Synthetic sequence
RREAIFCIQYNIRSFMNVKHWPW

SEQ ID NO: 29 - protein, 21 aa, Artificial sequence
  Synthetic sequence
EAIFCIQYNIRSFMNVKHWPW

SEQ ID NO: 30 - protein, 25 aa, Artificial sequence
  Synthetic sequence
VERREAIFCIQYNIRSFMNVKHWPW

SEQ ID NO: 31 - protein, 27 aa, Artificial sequence
  Synthetic sequence
RMVERREAIFCIQYNIRSFMNVKHWPW

SEQ ID NO: 32 - protein, 29 aa, Artificial sequence
  Synthetic sequence
YQRMVERREAIFCIQYNIRSFMNVKHWPW

SEQ ID NO: 33 - protein, 31 aa, Artificial sequence
  Synthetic sequence
VEYQRMVERREAIFCIQYNIRSFMNVKHWPW

SEQ ID NO: 34 - protein, 33 aa, Artificial sequence
  Synthetic sequence
ARVEYQRMVERREAIFCIQYNIRSFMNVKHWPW

SEQ ID NO: 35 - protein, 33 aa, Artificial sequence; identical to SEQ ID NO: 34
  Synthetic sequence

SEQ ID NO: 36 - protein, 35 aa, Artificial sequence
  Artificial sequence
FLARVEYQRMVERREAIFCIQYNIRSFMNVKHWPW

SEQ ID NO: 37 - protein, 37 aa, Artificial sequence
  Synthetic sequence
RGFLARVEYQRMVERREAIFCIQYNIRSFMNVKHWPW

SEQ ID NO: 38 - protein, 22 aa, Artificial sequence
  Synthetic sequence
AIFCIQYNIRSFMNVKHWPWMK

SEQ ID NO: 39 - protein, 24 aa, Artificial sequence
  Synthetic sequence
AIFCIQYNIRSFMNVKHWPWMKLF

SEQ ID NO: 40 - protein, 26 aa, Artificial sequence
  Synthetic sequence
AIFCIQYNIRSFMNVKHWPWMKLFFK

SEQ ID NO: 41 - protein, 28 aa, Artificial sequence
  Synthetic sequence
AIFCIQYNIRSFMNVKHWPWMKLFFKIK

SEQ ID NO: 42 - protein, 30 aa, Artificial sequence
  Synthetic sequence
AIFCIQYNIRSFMNVKHWPWMKLFFKIKPL

SEQ ID NO: 43 - protein, 32 aa, Artificial sequence
  Synthetic sequence
AIFCIQYNIRSFMNVKHWPWMKLFFKIKPLLK

SEQ ID NO: 44 - protein, 34 aa, Artificial sequence
  Synthetic sequence
AIFCIQYNIRSFMNVKHWPWMKLFFKIKPLLKSA

SEQ ID NO: 45 - protein, 36 aa, Artificial sequence
  Synthetic sequence
AIFCIQYNIRSFMNVKHWPWMKLFFKIKPLLKSAET

SEQ ID NO: 46 - protein, 31 aa, Artificial sequence
  Synthetic sequence
SQEEIKNMWAAFPPDVGGNVDYKNICYVITH

SEQ ID NO: 47 - protein, 32 aa, Artificial sequence
  Synthetic sequence
SQEEIKNMWAAFPPDVGGNVDYKNICYVITHG

SEQ ID NO: 48 - protein, 33 aa, Artificial sequence
  Synthetic sequence
SQEEIKNMWAAFPPDVGGNVDYKNICYVITHGD

SEQ ID NO: 49 - protein, 34 aa, Artificial sequence
  Synthetic sequence
SQEEIKNMWAAFPPDVGGNVDYKNICYVITHGDA

SEQ ID NO: 50 - protein, 35 aa, Artificial sequence
  Synthetic sequence
SQEEIKNMWAAFPPDVGGNVDYKNICYVITHGDAK

SEQ ID NO: 51 - protein, 33 aa, Artificial sequence
  Synthetic sequence
FSQEEIKNMWAAFPPDVGGNVDYKNICYVITHG

SEQ ID NO: 52 - protein, 34 aa, Artificial sequence
  Synthetic sequence
FSQEEIKNMWAAFPPDVGGNVDYKNICYVITHGD

SEQ ID NO: 53 - protein, 35 aa, Artificial sequence
  Synthetic sequence
FSQEEIKNMWAAFPPDVGGNVDYKNICYVITHGDA

SEQ ID NO: 54 - protein, 36 aa, Artificial sequence
  Synthetic sequence
FSQEEIKNMWAAFPPDVGGNVDYKNICYVITHGDAK

SEQ ID NO: 55 - protein, 34 aa, Artificial sequence
  Synthetic sequence
RFSQEEIKNMWAAFPPDVGGNVDYKNICYVITHG

SEQ ID NO: 56 - protein, 35 aa, Artificial sequence
  Synthetic sequence
RFSQEEIKNMWAAFPPDVGGNVDYKNICYVITHGD

SEQ ID NO: 57 - protein, 36 aa, Artificial sequence
  Synthetic sequence
RFSQEEIKNMWAAFPPDVGGNVDYKNICYVITHGDA

SEQ ID NO: 58 - protein, 37 aa, Artificial sequence
  Synthetic sequence
RFSQEEIKNMWAAFPPDVGGNVDYKNICYVITHGDAK

SEQ ID NO: 59 - protein, 35 aa, Artificial sequence
  Synthetic sequence
DRFSQEEIKNMWAAFPPDVGGNVDYKNICYVITHG

SEQ ID NO: 60 - protein, 36 aa, Artificial sequence
  Synthetic sequence
DRFSQEEIKNMWAAFPPDVGGNVDYKNICYVITHGD

SEQ ID NO: 61 - protein, 37 aa, Artificial sequence
  Synthetic sequence
DRFSQEEIKNMWAAFPPDVGGNVDYKNICYVITHGDA

SEQ ID NO: 62 - protein, 38 aa, Artificial sequence
  Synthetic sequence
DRFSQEEIKNMWAAFPPDVGGNVDYKNICYVITHGDAK

SEQ ID NO: 63 - protein, 32 aa, Artificial sequence
  Synthetic sequence
FSQEEIKNMWAAFPPDVGGNVDYKNICYVITH

SEQ ID NO: 64 - protein, 33 aa, Artificial sequence
  Synthetic sequence
RFSQEEIKNMWAAFPPDVGGNVDYKNICYVITH

SEQ ID NO: 65 - protein, 34 aa, Artificial sequence
  Synthetic sequence
DRFSQEEIKNMWAAFPPDVGGNVDYKNICYVITH

SEQ ID NO: 66 - protein, 31 aa, Artificial sequence
  Synthetic sequence
QEEIKNMWAAFPPDVGGNVDYKNICYVITHG

SEQ ID NO: 67 - protein, 32 aa, Artificial sequence
  Synthetic sequence
QEEIKNMWAAFPPDVGGNVDYKNICYVITHGD

SEQ ID NO: 68 - protein, 33 aa, Artificial sequence
  Artificial sequence
QEEIKNMWAAFPPDVGGNVDYKNICYVITHGDA

SEQ ID NO: 69 - protein, 34 aa, Artificial sequence
  Synthetic sequence
QEEIKNMWAAFPPDVGGNVDYKNICYVITHGDAK

SEQ ID NO: 70 - protein, 30 aa, Artificial sequence; identical to SEQ ID NO: 15
  Synthetic sequence

SEQ ID NO: 71 - protein, 28 aa, Artificial sequence
  Synthetic sequence
EQFLPMMQAISNNKDQATYEDFVEGLRV

SEQ ID NO: 72 - protein, 28 aa, Artificial sequence
  Synthetic sequence
EQFLPMMQAISNNKDQATYEDFVEGLRV

SEQ ID NO: 73 - protein, 27 aa, Artificial sequence
  Synthetic sequence
QFLPMMQAISNNKDQATYEDFVEGLRV

SEQ ID NO: 74 - protein, 26 aa, Artificial sequence
  Synthetic sequence
FLPMMQAISNNKDQATYEDFVEGLRV

SEQ ID NO: 75 - protein, 25 aa, Artificial sequence
  Synthetic sequence
LPMMQAISNNKDQATYEDFVEGLRV

SEQ ID NO: 76 - protein, 25 aa, Artificial sequence
  Artificial sequence
LPMMQAISNNKDQATYEDFVEGLRV

SEQ ID NO: 77 - protein, 23 aa, Artificial sequence
  Synthetic sequence
MMQAISNNKDQATYEDFVEGLRV

SEQ ID NO: 78 - protein, 22 aa, Artificial sequence
  Synthetic sequence
MQAISNNKDQATYEDFVEGLRV

SEQ ID NO: 79 - protein, 21 aa, Artificial sequence
  Synthetic sequence
QAISNNKDQATYEDFVEGLRV

SEQ ID NO: 80 - protein, 20 aa, Artificial sequence
  Synthetic sequence
AISNNKDQATYEDFVEGLRV

SEQ ID NO: 81 - protein, 19 aa, Artificial sequence
  Synthetic sequence
ISNNKDQATYEDFVEGLRV

SEQ ID NO: 82 - protein, 18 aa, Artificial sequence
  Synthetic sequence
SNNKDQATYEDFVEGLRV

SEQ ID NO: 83 - protein, 17 aa, Artificial sequence
  Synthetic sequence
NNKDQATYEDFVEGLRV

SEQ ID NO: 84 - protein, 16 aa, Artificial sequence
  Synthetic sequence
NKDQATYEDFVEGLRV

SEQ ID NO: 85 - protein, 15 aa, Artificial sequence
  Synthetic sequence
KDQATYEDFVEGLRV

SEQ ID NO: 86 - protein, 14 aa, Artificial sequence
  Synthetic sequence
DQATYEDFVEGLRV

SEQ ID NO: 87 - protein, 13 aa, Artificial sequence
  Synthetic sequence
QATYEDFVEGLRV

SEQ ID NO: 88 - protein, 12 aa, Artificial sequence
  Synthetic sequence
ATYEDFVEGLRV

SEQ ID NO: 89 - protein, 11 aa, Artificial sequence
  Synthetic sequence
TYEDFVEGLRV

SEQ ID NO: 90 - protein, 11 aa, Artificial sequence
  Synthetic sequence
YEDFVEGLRVF

SEQ ID NO: 91 - protein, 12 aa, Artificial sequence
  Synthetic sequence
YEDFVEGLRVFD

SEQ ID NO: 92 - protein, 13 aa, Artificial sequence
  Synthetic sequence
YEDFVEGLRVFDK

SEQ ID NO: 93 - protein, 13 aa, Artificial sequence
  Synthetic sequence
YEDFVEGLVFDKE

SEQ ID NO: 94 - protein, 14 aa, Artificial sequence
  Synthetic sequence
YEDFVEGLVFDKEG

SEQ ID NO: 95 - protein, 15 aa, Artificial sequence
  Synthetic sequence
YEDFVEGLVFDKEGN

SEQ ID NO: 96 - protein, 16 aa, Artificial sequence
  Synthetic sequence
YEDFVEGLVFDKEGNG

SEQ ID NO: 97 - protein, 22 aa, Artificial sequence
  Synthetic sequence
AIFCIQYNIRSFMNVKHWPWMK

SEQ ID NO: 98 - protein, 26 aa, Artificial sequence
  Synthetic sequence
  MOD_RES (11)..(11): X is unknown residue
  MOD_RES (13)..(13): L is Norleucine
  MOD_RES (14)..(14): N is N-Methyl asparagine
  MOD_RES (21)..(21): L is Norleucine
AIFSIQYNIRXFLNVKHWPWLKKKKK